(19) United States
(12) Patent Application Publication
(10) Pub. No.: US 2009/0068143 A1
(43) Pub. Date: Mar. 12, 2009

(54) ORALLY EFFECTIVE CANNABINOID ANALOGS

(75) Inventors: Avihai Yacovan, Gedera (IL); Avi Bar-Joseph, Rehovot (IL); Sigal Meilin, Givatayim (IL); Shimon Amselem, Rehovot (IL)

Correspondence Address: FENNEMORE CRAIG, 3003 NORTH CENTRAL AVENUE, SUITE 2600, PHOENIX, AZ, 85012, US

(73) Assignee: PHARMOS CORPORATION, Iselin, NJ (US)

(21) Appl. No.: 11/577,349

(22) PCT Filed: Feb. 24, 2005
(86) PCT No.: PCT/IL05/00231

§ 371 Date: Jul. 6, 2007

(51) Int. Cl. A61K 31/192; A61K 31/122; A61K 38/21; A61K 31/7076; A61K 31/573; A61P 29/00; A61P 25/00; A61K 31/675; A61K 31/52; A61K 31/235

(52) U.S. Cl. 424/85.6; 514/680; 514/548; 514/569; 514/263.2; 514/45; 514/110; 514/171

(57) ABSTRACT

The present invention relates to orally effective ligands of the peripheral cannabinoid receptor CB₂, especially (+)-α-pinene derivatives, and to pharmaceutical compositions thereof, which are useful for prevention, alleviation or treatment of autoimmune neurodegenerative disorders, in particular multiple sclerosis and associated symptoms. Methods of the invention are useful when the active ingredient is administered alone or in combination with existing therapeutic modalities. The compositions are administered by oral route.

### FIELD OF THE INVENTION

The present invention relates to orally effective ligands of the peripheral cannabinoid receptor CB₂, especially (+)-α-pinene derivatives, and to pharmaceutical compositions thereof, which are useful for prevention, alleviation or treatment of autoimmune neurodegenerative disorders, in particular multiple sclerosis.

### BACKGROUND OF THE INVENTION

Cannabis was historically used for the treatment of insomnia, inflammation, pain, various psychoses, digestive disorders, depression, migraine, neuralgia, fatigue, constipation, diarrhea, parasites, infections and appetite disorders. Some of the potential medical uses of cannabis have generated voluminous scientific literature reviewed by Pate [Pate D. W., Journal of the International Hemp Association 2(2): 74-6, 1995]. There is a growing amount of evidence suggesting that cannabis, and its individual bioactive components the cannabinoids, may be effective in suppressing certain symptoms of multiple sclerosis and spinal cord injury. Cannabis comprises about 60 different cannabinoids such as cannabinol (CBN), cannabidiol (CBD), cannabichromene (CBC) and cannabigerol (CBG). Cannabinoids are hydrophobic compounds that exert most of their actions via the activation of specific G-protein coupled receptors. To date, two cannabinoid receptors have been cloned and characterized, cannabinoid type 1 receptor (CB₁) and cannabinoid type 2 receptor (CB₂), although additional receptors may exist. The CB₁ receptors are predominantly found in the central nervous system (CNS) and are responsible for the psychotropic effects of cannabinoids, whereas the CB₂ receptors are expressed mainly in the periphery on immune cells.

The major psychoactive constituent of cannabis is Δ⁹-tetrahydrocannabinol (THC) and it is one of the rare cannabinoids to be approved for use in medicine. Dronabinol is a synthetic THC encapsulated in sesame oil sold under the trade name of Marinol® as a schedule II controlled substance. It has been accepted in the United States as an anti-emetic to treat the nausea associated with cancer chemotherapy since 1985 and as an appetite stimulant in AIDS patients since 1992. Other clinical applications of cannabinoids have been reviewed by Robson [Robson P. British Journal of Psychiatry 178: 107-115, 2001].

Clinical trials, performed on small number of patients, indicate that, THC can produce objective and/or subjective relief from spasticity, pain, tremor, bladder related symptoms and nocturia in patients with MS. It has been suggested that the lack of more significant evidence that THC may be effective in MS may be due to the oral route of administration employed. It is believed that some of these beneficial cannabinoid-induced activities are mediated by the two identified cannabinoid receptors CB₁ and CB₂. However, the activity in MS of the non-psychoactive cannabidiol (CBD), which does not bind to either known cannabinoid receptors, indicates that part of the therapeutic effects might be through non-receptor mediated mechanism. It should be stressed that the results of these clinical trials have been considered equivocal by some [Killestein J. et al., Neurology 58: 1404-7, 2002].

THC is currently being given orally, but is often ineffective when taken in this form due to low and erratic bioavailability. For example, a meta-analysis study revealed a poor or only partial response to THC in approximately 65% of 750 courses of oral therapy. Thus, high single doses are administered which may cause undesirable side effects such as sedation, confusion and anxiety. Such poor response to oral administration of THC may be due to the limited aqueous solubility of THC, its extensive first pass metabolism following oral administration, and the resulting low absolute bioavailability of THC (13% on an average). Previous studies have also reported that another limitation of orally administered THC is the large inter-subject variability in absorption. Russo [Russo E. B., Neurology 60(4): 729, 2003] reported that the symptomatic improvement experienced by MS patients was significant with orally ingested, smoked or vaporized cannabis, whereas the efficacy of oral Marinol® was dubious.

It would be advantageous to obtain cannabinoids with increased water solubility, which might lead to the preparation of orally available medicines with stable and potentially higher bioavailability. The advantages of oral administration over other routes are numerous and include first and foremost patient compliance and safety.

Multiple Sclerosis (MS) is an inflammatory disease of the CNS which affects the brain and the spinal cord and it is the most common cause of neurological disability in young adults. Predominantly, it is a disease caused by demyelination of the nerve fibers, primarily in the white matter, believed to result from chronic inflammation of the CNS, but other forms of nerve degeneration have been reported. The integrity of the myelin sheath covering the axon and ensuring proper transmission of nerve impulses is maintained by oligodendrocytes, which belong to a larger group of maintenance cells called glial cells. It seems that oligodendrocyte loss precedes inflammation. As the disease progresses, axons are less destroyed by the inflammatory process and more by Wallerian degeneration following distal injury to the same axon. Many processes contribute therefore to the symptoms of MS during the progression of the disease and they include inflammation, demyelination, oligodendrocyte death, membrane damage and axonal death. It is generally considered that MS has two etiologic phases, a first autoimmune trigger followed by neurodegeneration.

The treatment of MS generally falls into two categories: treatments that address symptom management, and treatments that change the course of the disease by modifying the number and severity of attacks and the progression of disability. Five different products have been approved by the FDA as disease modifying agents (DMA) for the treatment of MS since 1993. These included three interferon-beta (IFN-β) products (Betaseron®, Avonex®, and Rebif®), which are immunomodulators, and two unrelated products (Copaxone® and Novantrone®).

All existing disease modifying treatments cause mild to severe side-effects well known to medical practitioners, including flu-like symptoms, liver toxicity, transient flushing, chest and joint pains, weakness, nausea, anxiety, muscle stiffness, cardiotoxicity and potential leukaemogenicity. More importantly, the protein-based therapies often elicit neutralizing antibodies (NAbs) which ultimately annihilate the efficacy of the drug with continued use. More common problems are injection site reactions and the difficulties many people have injecting themselves.

The DMA used in MS therapy, either immunomodulators or immunosuppressors, target the immune components of the disease. However, as previously detailed MS can produce a wide range of symptoms which can be classified as visual, motor, sensory, coordination and balance, bowel, bladder, sexual, cognitive and others. The symptomatic treatments include the administration of steroids, anti-convulsants, tricyclic antidepressants, anti-inflammatory drugs, non-steroidal anti-inflammatory drugs (NSAID), selective serotonin reuptake inhibitors (SSRI), monoamine oxidase inhibitors (MOI), antidepressants, benzodiazepines (BZD), muscle relaxants, anticholinergic agents, beta-blockers, laxatives, and some specific channel blockers. Combination therapies with immunomodulators, antioxidant, and neuroprotective drugs are currently being investigated. Treatment with drugs that might enhance the remyelination of lesion sites is also being considered and could be included in future combination therapies. It would be advantageous if a single agent could address more than one of the numerous MS associated symptoms and act on its own as multifactorial therapy.

Though the treatment of MS has changed dramatically in the last decade, the existing medications mentioned above still have few drawbacks. The DMA are all injectable medicaments causing low patient compliance and frequent injection site reactions. Neutralizing antibodies can appear against IFN-β and Copaxone® and they may reduce treatment efficacy. Multiple therapeutic agents are needed in addition to address the various symptoms associated with MS and these drugs are themselves not devoid of side effects. The annual cost for MS treatment may further rise with accumulation of additional symptomatic treatments.

It would be advantageous to obtain an orally administrable medicine in order to gain a higher medicinal compliance ratio, a lower need to attend hospital, and therefore a higher quality of life of the patient. It would be a further advantage if such medicine would alleviate or treat simultaneously a number of symptoms. Small molecules are usually cheaper to prepare than peptides and proteins and have in addition less susceptibility to elicit neutralizing antibody, even following chronic administration. It would be an additional improvement for the treatment of multiple sclerosis, if the existing biological DMA could be replaced by relatively small, safer and cheaper chemical entities.

Cannabinoids are known to have neuroprotective properties and can regulate glutamate release and oxidative free radicals that may additionally contribute to MS. It has been recently suggested that cannabinoids might even prevent demyelination and/or enhance remyelination [Arevalo-Martin A. et al., Journal of Neuroscience 23(7): 2511-6, 2003]. Moreover, it is known that cannabinoids have immunomodulator properties and therefore they may have effects not only on the symptoms, but also on the onset and development of MS. Owing to their wide range of therapeutic activity, cannabinoids might advantageously replace the existing costly DMA presently used for the treatment of MS as well as other symptomatic medications. The evidences supporting use of cannabinoids in MS have been recently reviewed [Atha M. J., IDMU Literature Review: 1-11, 2002].

It is know that CB₂ selective cannabinoids, which were credited for the immunomodulatory activity of cannabinoids, are considered less psychoactive, if at all, than the naturally occurring THC. It would be therefore advantageous to develop CB₂ selective cannabinoids for the treatment or alleviation of MS and it would be additionally beneficial if said compounds would be water soluble and orally bioavailable.

Numerous patents and published applications are directed to orally available treatment for multiple sclerosis. Among them, U.S. Pat. No. 4,994,466 discloses the use of narcotic antagonist for the treatment of MS by oral administration, U.S. Pat. No. 5,217,958 teaches the use of phytic acid, U.S. Pat. No. 5,869,054 discloses a slightly different approach wherein the oral agent is an autoantigen in particular myelin base protein (MBP) or fragments thereof, whereas International Patent Application WO 95/27500 describes other agents to achieve oral tolerization. US Patent Application 2004/086534 discloses the use of IFN-τ for the treatment of MS by oral administration, International Patent Application WO 98/30227 the use of COP-1, and International Patent Application WO 00/09127 the use of Idublast. None of the above mentioned agents are cannabinoids.

Other patents more specifically disclosed the use of cannabinoid agonists or antagonists for the treatment of MS. For instance for the treatment of multiple sclerosis U.S. Pat. No. 5,618,955 discloses the use of polyunsaturated fatty acid amides and their derivatives, US Patent Application 2004/0186166 the use of cannabinoid analogs such as alujemic acid, US Patent Application 2004/0157823 the use of 3-aminoazetidine derivatives, US Patent Application 2004/0138293 the use of cannabis extract, US Patent Application 2004/0132804 the use of amino indanes derivatives, US Patent Application 2004/0116326 the use of 1,3-thiazine derivatives, US Patent Application 2004/0110827 the use of enantiomerically pure dexanabinol, US Patent Application 2004/0106800 the use of 4,5-dihydro-1H-pyrazole derivatives and US Patent Application 2004/0106614 the use of 1H-1,2,4-triazole-3-carboxamide derivatives. The compounds disclosed in these applications are not necessarily cannabinoid agonists nor CB₂ selective and they are generally structurally distinct from α-pinene derivatives of the present invention.

U.S. Pat. No. 4,208,351 discloses optically active bicyclic compounds as intermediates in a stereoselective process for the preparation of classical tricyclic cannabinoids. However, no therapeutic activity was attributed to the intermediates, no mention was made to the ability of such compounds to bind cannabinoid receptors altogether and thus no pharmaceutical composition comprising such compounds were envisioned.

U.S. Pat. No. 4,282,248 discloses both isomeric mixtures and individual isomers of pinene derivatives. Therapeutic activity, including analgesic, central nervous system depressant, sedative and tranquilizing activity, was attributed to the compounds, but the disclosure did not teach that said compounds would bind to any cannabinoid receptor.

U.S. Pat. No. 5,434,295 discloses a family of novel 4-phenyl pinene derivatives, and teaches how to utilize said compounds in pharmaceutical compositions useful in treating various pathological conditions associated with damage to the central nervous system. This disclosure neither teaches nor suggests that any of those are selective for peripheral cannabinoid receptors.

International patent application WO 01/28497 discloses novel bicyclic cannabinoid analogs that exhibit high affinity for the CB₂ receptor. It is apparent to the skilled artisan that the compounds in said application are (−) α-pinene derivatives and therefore of a stereochemical orientation wherein C-1, C-4 and C-5 are R, when referring to the nomenclature adopted in the present disclosure. This application suggests that (−) α-pinene derivatives could be useful in the treatment of MS. The only information disclosed concerning the sole compound of the application relates to binding activity in vitro toward CB₁ and CB₂.

International patent application WO 01/32169 discloses a family of (+) α-pinene bicyclic compounds, including HU-308, as CB₂ specific agonists and exemplifies their use in the treatment of pain and inflammation, autoimmune diseases, gastrointestinal disorders and as hypotensive agents. This application suggests that (+) α-pinene derivatives could be useful in the treatment of MS.

International patent application WO 03/005960 discloses novel cannabinoid analogs that exhibit high affinity for the CB₂ receptor, some of them of bicyclic structure. This application claims both the (−) and (+) enantiomers and all isomers of pinene derivatives. The only bicyclic pinene compounds disclosed are derived from (−) α-pinene and the sole biological information relates to binding activity in vitro. This application suggests that any pinene derivative could be useful in the treatment of MS, but it does not demonstrate that said compounds are indeed effective in vivo in any disease model.

International patent application WO 03/063758 discloses novel (+) α-pinene derivatives that exhibit selectivity for the CB₂ receptor. This application discloses certain hydrophilic bicyclic cannabinoids and demonstrates that such compounds are among other things effective anti-inflammatory and analgesic agents when administered parenterally. This application disclosed that hydrophobic compounds of the invention are useful in the treatment of MS when administered intravenously, and suggested that certain of these compounds might be delivered orally.

Currently, the drugs used for alleviating or treating multiple sclerosis suffer from certain shortcomings. It would be advantageous to develop orally available small molecules, with better safety profiles, patient compliance and lower cost. Cannabinoids provide candidates for the treatment of MS. This class of compounds has the potential added advantage to both address the pivotal immune component of the disease and relief MS associated symptoms. Thus, the present invention provides solutions to the long-felt unmet medical need for therapeutic means of intervening in multiple sclerosis, and other disorders having autoimmune and neurodegenerative etiology.

### SUMMARY OF THE INVENTION

The present invention overcomes the deficiencies of the background art by providing cannabinoids which are orally effective and prevent, alleviate or treat disorders having an autoimmune and a neurodegenerative etiology, in particular multiple sclerosis.

The orally effective cannabinoids of the invention are useful in the treatment of multiple sclerosis associated symptoms. It is believed that the orally effective compositions of the invention are useful to treat or prevent these neurological symptoms in a variety of disorders of the nervous system other than multiple sclerosis. Thus, the orally effective compositions are useful to treat or prevent tremor, spasticity, muscle weakness, and lack of coordination of any etiology.

Specifically, the cannabinoids of the invention are CB₂ selective (+) α-pinene derivatives which are preferably water soluble. The compounds of the invention can be used to alleviate or treat multiple sclerosis or associated symptoms either alone, or in combination with other cannabinoids or with other medications used in the treatment of said disorders.

According to a first aspect, the present invention provides a method of preventing, alleviating or treating multiple sclerosis comprising the step of administering to an individual in need thereof an orally effective amount of a pharmaceutical composition comprising as an active ingredient a compound of formula (I):

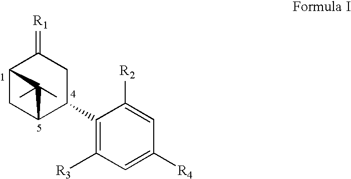

having a specific stereochemistry wherein C-4 is S, the protons at C-1 and C-5 are cis in relation to one another and the protons at C-4 and C-5 are trans; and wherein:
R₁ is selected from the group consisting of
(a)O or S,
(b) C(R′)₂ wherein R′ at each occurrence is independently selected from the group consisting of hydrogen, cyano, —OR″, —N(R″)₂, a saturated or unsaturated, linear or branched C₁-C₆ alkyl, C₁-C₆ alkyl-OR″ or C₁-C₆ alkyl-N(R″)₂ wherein at each occurrence R″ is independently selected from the group consisting of hydrogen, C(O)R′″, C(O)N(R′″)₂, C(S)R′″, saturated or unsaturated, linear or branched C₁-C₆ alkyl, C₁-C₆ alkyl-OR′″, and C₁-C₆ alkyl-N(R′″)₂, wherein at each occurrence R′″ is independently selected from the group consisting of hydrogen or saturated or unsaturated, linear, branched or cyclic C₁-C₁₂ alkyl, and
(c) NR″ or N—OR″ wherein R″ is as previously defined;
   R₂ and R₃ are each independently selected from the group consisting of
(a) —R″, —OR″, —N(R″)₂, —SR″, —S(O)(O)NR″, wherein at each occurrence R″ is as previously defined,
(b) —S(O)R^{b}, —S(O)(O)R^{b} wherein R^{b} is selected from the group consisting of hydrogen, saturated or unsaturated, linear or branched C₁-C₆ alkyl, C₁-C₆ alkyl-OR″, and C₁-C₆ alkyl-N(R″)₂, wherein R″ is as previously defined, and
(c) —OC(O)OH, —OS(O)(O)OR^{e}, —OP(O)(OR^{e})₂, —OR^{d} or —OC(O)—R^{d} chain terminated by —C(O)OH, —S(O)(O)OR^{e}, or —P(O)(OR^{e})₂, wherein R^{d} is a saturated or unsaturated, linear or branched C₁-C₆ alkyl and R^{e} is at each occurrence selected from the group consisting of hydrogen and R^{d} as previously defined; and
   R₄ is selected from the group consisting of
(a) R wherein R is selected from the group consisting of hydrogen, halogen, OR′″, OC(O)R′″, C(O)OR′″, C(O)R′″, OC(O)OR′″, CN, N(R′″)₂, NC(O)R′″, NC(O)OR′″, C(O)N(R′″)₂, NC(O)N(R′″)₂, and SR′″, wherein at each occurrence R′″ is as previously defined,
(b) a saturated or unsaturated, linear, branched or cyclic C₁-C₁₂ alkyl-R wherein R is as previously defined,
(c) an aromatic ring which can be further substituted at any position by R wherein R is as previously defined, and
(d) a saturated or unsaturated, linear, branched or cyclic C₁-C₁₂ alkyl optionally terminated by an aromatic ring which can be further substituted as defined in (c);

and pharmaceutically acceptable salts, esters or solvates thereof.

According to certain embodiments, the present invention provides a method of preventing, alleviating or treating multiple sclerosis, comprising the step of administering to an individual in need thereof an orally effective amount of a pharmaceutical composition comprising as an active ingredient a compound of formula (I) wherein R₁ is O, R₂ and R₃ are each OR^{f} wherein at each occurrence R^{f} is independently selected from the group consisting of hydrogen, —R^{d} and —C(O)—R^{d}, wherein R^{d} is a saturated or unsaturated, linear or branched C₁-C₆ alkyl chain terminated by —C(O)OR^{g} and R^{g} is selected from the group consisting of hydrogen and a saturated or unsaturated, linear or branched C₁-C₆ alkyl, and R₄ is selected from the group consisting of a saturated or unsaturated, linear, branched or cyclic C₁-C₁₂ alkyl-R^{h} wherein R^{h} is selected from the group consisting of R and an aromatic ring which can be optionally further substituted at any position by R as previously defined.

According to additional embodiments, the present invention provides a method of preventing, alleviating or treating multiple sclerosis, comprising the step of administering to an individual in need thereof an orally effective amount of a pharmaceutical composition comprising as an active ingredient a compound of formula (I) wherein R₁ is O, R₂ and R₃ are each independently selected from the group consisting of OH, succinate, fumarate, and methylenoxycarboxyl, and R₄ is selected from the group consisting of 1,1-dimethylpentyl, 1,1-dimethylheptyl, 1,1-dimethyl-6-heptynyl, 1,1-dimethyl-3-phenyl-propyl, 1,1,3-trimethyl-butyl, 1-(4-chloro-phenyl)-1-methyl-ethyl, 1-ethyl-1-methyl-propyl, 5-bromo-1,1-dimethyl-pentyl and 1,1-dimethyl-pent-4-enyl.

According to an exemplary embodiment, the present invention provides a method of preventing, alleviating or treating multiple sclerosis, comprising the step of administering to an individual in need thereof an orally effective amount of a pharmaceutical composition comprising as an active ingredient a compound of formula (I) wherein R₁ is O, R₂ is OH, R₃ is fumarate and R₄ is 1,1-dimethylheptyl.

According to another aspect, the present invention provides a method of preventing, alleviating or treating neurological symptoms selected from the list consisting of tremor, spasticity, muscle weakness, and lack of coordination, comprising the step of administering to an individual in need thereof an orally effective amount of a pharmaceutical composition comprising as an active ingredient a compound of formula (I) as defined above.

According to another aspect, the present invention provides a method of modulating mediators of inflammation comprising the step of administering to an individual in need thereof an orally effective amount of a pharmaceutical composition comprising as an active ingredient a compound of formula (I) as defined above.

According to another aspect, the present invention provides the use for the preparation of a medicament for preventing, alleviating or treating multiple sclerosis of an orally effective compound of formula (I) as defined above.

According to certain embodiments, the present invention provides the use for the preparation of a medicament for preventing, alleviating or treating multiple sclerosis of an orally effective compound of formula (I) wherein R₁ is O, R₂ and R₃ are each OR^{f} wherein at each occurrence R^{f} is independently selected from the group consisting of hydrogen, —R^{d} and —C(O)—R^{d}, wherein R^{d} is a saturated or unsaturated, linear or branched C₁-C₆ alkyl chain terminated by —C(O)OR^{g} and R^{g} is selected from the group consisting of hydrogen and a saturated or unsaturated, linear or branched C₁-C₆ alkyl, and R₄ is selected from the group consisting of a saturated or unsaturated, linear, branched or cyclic C₁-C₁₂ alkyl-R^{h} wherein R^{h} is selected from the group consisting of R and an aromatic ring which can be optionally further substituted at any position by R as previously defined.

According to additional embodiments, the present invention provides the use for the preparation of a medicament for preventing, alleviating or treating multiple sclerosis of an orally effective compound of formula (I) wherein R₁ is O, R₂ and R₃ are each independently selected from the group consisting of OH, succinate, fumarate, and methylenoxycarboxyl, and R₄ is selected from the group consisting of 1,1-dimethylpentyl, 1,1-dimethylheptyl, 1,1-dimethyl-6-heptynyl, 1,1-dimethyl-3-phenyl-propyl, 1,1,3-trimethyl-butyl, 1-(4-chloro-phenyl)-1-methyl-ethyl, 1-ethyl-1-methyl-propyl, 5-bromo-1,1-dimethyl-pentyl and 1,1-dimethyl-pent-4-enyl.

According to an exemplary embodiment, the present invention provides the use for the preparation of a medicament for preventing, alleviating or treating multiple sclerosis of an orally effective compound of formula (I) wherein R₁ is O, R₂ is OH, R₃ is fumarate and R₄ is 1,1-dimethylheptyl.

According to another aspect, the present invention provides the use for the preparation of a medicament for preventing, alleviating or treating neurological symptoms as previously defined, of an orally effective compound of formula (I) as defined above.

According to another aspect, the present invention provides the use for the preparation of a medicament for modulating mediators of inflammation of an orally effective compound of formula (I) as defined above.

Pharmaceutical compositions of the present invention may include in addition to orally effective compounds of formula (I), thickeners, carriers, buffers, diluents, surface active agents, preservatives and the like, all as well known in the art, necessary to produce a physiologically acceptable and stable formulation.

Pharmaceutical compositions may also include for the purpose of co-administration one or more additional active ingredients, such as, but not limited to compounds of formula (I), anti-inflammatory agents, immunomodulators, immunosuppressors, steroids, anti-convulsants, analgesics, anti-depressants, muscle relaxants, and the like, known to medical practitioners.

In the present specification and claims which follow, co-administration is explicitly meant to include combined therapies that are administered individually or as a single composition. When administered individually, the separate therapeutic agents may be administered at substantially the same time or under separate regimen.

The pharmaceutical compositions of the invention are administered orally for patient convenience, comfort and safety. The routes of administration include but are not limited to peroral, wherein the drug is swallowed, and buccal, gingival, lingual, sublingual and oro-pharyngeal administration for trans-mucosal absorption in the oral cavity.

The pharmaceutical compositions may be in a liquid, aerosol or solid dosage form, and may be formulated into any suitable formulation including, but not limited to, solutions, suspensions, micelles, emulsions, microemulsions, aerosols, powders, granules, sachets, soft gels, capsules, tablets, pills, caplets and the like, as will be required for the oral route of administration.

Prior to their use as medicaments for preventing, alleviating or treating an individual in need thereof, the pharmaceutical compositions may be formulated in unit dosage form. The active dose for humans is generally in the range of from 0.05 mg to about 50 mg per kg body weight, in a regimen of 1-4 times a day. However, it is evident to a person skilled in the art that the selected dosage of the active ingredient depends upon the desired therapeutic effect, the route of administration, the duration of treatment desired, the patient's age, weight, contraindications, co-administration and combination with additional medications and the like.

These and additional benefits and features of the invention could be better understood by those skilled in the art with reference to the following detailed description taken in conjunction with the figures and non-limiting examples.

### BRIEF DESCRIPTION OF THE FIGURES

The accompanying drawings, which are incorporated in and form a part of the specification, illustrate certain embodiments of the present invention, and together with the description serve to explain the principles of the invention. In the drawings:

FIG. 1 shows the effect of compound 18F administered p.o. on MBP induced acute EAE. Panel A displays the mean group clinical score along time. Panel B displays the mean maximal score per treatment group. Panel C displays the area under the curve per treatment group.

FIG. 2 shows the effect of compound 18F administered p.o. on PLP induced remitting-relapsing EAE.

FIG. 3 shows the effect of compound 18F administered p.o. on MOG induced chronic progressive EAE. Panel A displays the effect of various doses on clinical score along time and panel B shows the impact of control drugs and compound on disease progression.

FIG. 4 is a chromatogram of a western blot showing the effect of compounds 18A and 18F at various doses on iNOS protein expression in activated macrophages.

FIG. 5 shows the analgesic activity in visceral pain model as expressed by number of writhing response (WR). Panel A shows the effect of CB₁ and CB₂ antagonists on the analgesic activity of compounds 18A and 18F. Panel B shows the effect of combination therapy on analgesic activity. Panel C shows the effect of various doses of compound 18F administered p.o.

FIG. 6 shows the analgesic activity in visceral pain model as expressed by number of writhing response (WR). Panel A shows the effect of various compounds administered p.o. in cosolvent vehicle. Panel B shows the effect of various compounds administered p.o. in aqueous vehicle.

FIG. 7 shows the effect of various doses of compound 18F administered p.o. on inflammatory pain. Panel A displays the effect of various doses on paw volume as compared to vehicle treated animals. Panel B demonstrates the efficacy against thermal hyperalgesia and panel C against mechanical hyperalgesia.

FIG. 8 shows the effect of various doses of compound 18F administered p.o. on neuropathic pain. Panel A shows the reduction in pain response in chronic constriction induced neuropathic pain. Panel B displays the impact on mechanical hyperalgesia in Taxol® induced neuropathic pain.

### DETAILED DESCRIPTION OF THE INVENTION

The present invention provides compositions and methods for preventing, alleviating or treating disorders having autoimmune and neurodegenerative etiology using orally effective non-classical cannabinoids.

In particular the present invention provides pharmaceutical compositions comprising as an active ingredient CB₂ selective cannabinoid agonists and methods using the same for preventing, alleviating or treating multiple sclerosis.

Methods of the invention can be used to treat MS associated symptoms, whether caused by multiple sclerosis or resulting from other diseases or disorders.

Typically, the CB₂ selective agonist is a plant or animal derived cannabinoid or cannabimimetic compound selected from the group consisting of aminoalkylindoles, anandamides, 3-aroylindoles, aryl and heteroaryl sulfonates, arylsulphonamides, benzamides, biphenyl-like cannabinoids, cannabinoids optionally further substituted by fused or bridged mono- or polycyclic rings, pyrazole-4-carboxamides, eicosanoids, dihydroisoindolones, dihydrooxazoles, α-pinene derivatives, quinazolinediones, quinolinecarboxylic acid amides, resorcinol derivatives, tetrazines, triazines, pyridazines and pyrimidine derivatives, and analogues and derivatives thereof. More preferably, the CB₂ selective cannabinoid agonist is a α-pinene derivative, most preferably a (+)-α-pinene derivative.

### DEFINITIONS

To facilitate an understanding of the present invention, a number of terms and phrases are defined below.

As used herein, the term “central nervous system” (CNS) refers to all structures within the dura mater. Such structures include, but are not limited to, the brain and spinal cord.

As used herein, the term “CB” refers to cannabinoid receptors. CB₁ receptors are predominantly found in the CNS, whereas CB₂ receptors are predominantly found in the periphery on immune cells. Aside from these two receptors, evidence exists supporting the presence of yet uncloned cannabinoid receptors.

As used herein, the term “orally effective” indicates that compounds of the invention achieve the targeted biological activity in a subject following oral administration of the desired dosage in a reasonable volume.

As used herein, the term “water soluble” indicates that compounds of the invention dissolve in aqueous solutions better than Δ⁹-THC by at least 1 fold, preferably 50 folds, more preferably by 250 folds, and most preferably by 1000 folds or more.

In the present specification and claims which follow “inhibiting, reducing, or decreasing effect” is the ability to reduce the activity under discussion by at least 20%, preferably 40%, more preferably 60% and most preferably 80% or greater. In case of activities wherein the maximal possible effect is not 100%, the previous figures relate to percent of maximal possible effect.

In the present specification and claims which follow “enhancing or increasing effect” is the ability to increase the activity under discussion by at least 1.5 folds, preferably 3 folds, more preferably 4 folds and most preferably 5 folds or more.

In the present invention, binding affinity is represented by the IC₅₀ value, namely the concentration of a test compound that will displace 50% of a radiolabeled agonist from the CB receptors. Preferred compounds display IC₅₀ value for CB₂ binding of 50 nM or lower, preferably of 30 nM or lower, more preferably of 10 nM or lower and most preferably of 1 mM or lower. “CB₂ specific or selective” denotes compounds with a ratio of CB₂/CB₁ binding affinity that is at least 10, preferably 20, more preferably 30 and most preferably 50 or greater. Preferably these ratios will be obtained for human CB₁ and CB₂ receptors. The selectivity toward CB₂, denoted CB₂/CB₁ affinity, is calculated as the IC₅₀ value obtained by the test compound for the displacement of the CB₁ specific radioligand divided by the IC₅₀ value obtained for the displacement of the CB₂ specific radioligand, i.e. the IC₅₀ CB₁/IC₅₀ CB₂. Some of the preferred compounds of the present invention do not necessarily share both properties, in other words some have an IC₅₀ for CB₂ of about 1 nM but a ratio of only about 30.

An agonist is a substance that mimics a specific ligand, for example a hormone, a neurotransmitter, or in the present case a cannabinoid, able to attach to that ligand's receptor and thereby produce the same action that the natural ligand produces. Though most agonists act through direct binding to the relevant receptor and subsequent activation, some agonists act by promoting the binding of the ligand or increasing its time of residence on the receptor, increasing the probability and effect of each coupling. Whatever the mechanism of action, all encompassed in the present invention, the net effect of an agonist is to promote the action of the original chemical substance serving as ligand. Compounds that have the opposite effect, and instead of promoting the action of a ligand, block it are the receptor antagonists.

Though the most probable mechanism of action of compounds of the invention is through their selective binding to the CB₂ receptor and functional coupling to specific signal transduction pathways, alternative mechanisms cannot be ruled out, for instance either through binding to additional yet unidentified cannabinoid receptors or through non-receptor mediated means, or a combination of such mechanisms.

### Multiple Sclerosis

As used herein, the term “multiple sclerosis” or “MS” refers to an inflammatory disease of the central nervous system in genetically susceptible host. This neurodegenerative disease secondary to an autoimmune response primarily affects white matter tissue predominantly due to demyelination of the nerve fibers. Many processes contribute to the symptoms of MS during the progression of the disease and they include inflammation, demyelination, oligodendrocyte death, membrane damage and axonal death.

Clinically, MS is difficult to characterize because it is very unpredictable and variable. Depending on which areas of the CNS are affected and how badly they are damaged, the type and severity of symptoms can vary greatly. An optic nerve, lesion may cause blurred vision, a brain stem lesion may cause dizziness and a spinal cord lesion may cause coordination and/or balance problems. In general, people with MS can experience partial or complete loss of any function that is controlled by, or passes through, the brain or spinal cord.

Like other presumed autoimmune diseases, MS is more common in females with a gender ratio of about 2:1, and clinical symptoms often manifest during young adulthood. MS is predominantly a disease of temperate latitudes and of the western hemisphere. It is mainly reported in Europe, North America, Australia and New Zealand and in these regions its incidence can be as high as 250 per 100,000. Although MS is found in Japan, China and other temperate eastern countries, it is much rarer than in the West.

There are four main varieties of MS as defined in an international survey of neurologists [Lubin F. D. and Reingold S. C, Neurology 46(4): 907-11, 1996]. Relapsing/Remitting (RRMS) is characterized by relapses (also known as exacerbations) during which time new symptoms can appear and old ones resurface or worsen. The relapses are followed by periods of remission, during which time the person fully or partially recovers from the deficits acquired during the relapse. Relapses can last for days, weeks or months and recovery can be slow and gradual or almost instantaneous. The vast majority of people presenting with Multiple Sclerosis are first diagnosed with relapsing/remitting.

After a number of years many people who have had relapsing/remitting MS will pass into a secondary progressive phase of the disease (SPMS). This is characterized by a gradual worsening of the disease between relapses. In the early phases of Secondary Progressive, the person may still experience a few relapses but after a while these merge into a general progression. People with secondary progressive may experience good and bad days or weeks, but, apart from some remission following relapsing episodes, no real recovery. After 10 years, 50% of people with relapsing/remitting MS will have developed secondary progressive [Weinshenker B. G. et al, Brain 112: 133-46, 1989]. By 25 to 30 years, that figure will have risen to 90%.

A third form of the disease is known as Progressive Relapsing Multiple Sclerosis (PRMS). This form of MS follows a progressive course from onset, punctuated by relapses. There is significant recovery immediately following a relapse but between relapses there is a gradual worsening of symptoms.

Finally, the Primary Progressive (PPMS) type of MS is characterized by a gradual progression of the disease from its onset with no remissions at all. There may be periods of a leveling off of disease activity and, as with secondary progressive, there may be good and bad days or weeks. PPMS differs from Relapsing/Remitting and Secondary Progressive in that onset is typically in the late thirties or early forties, men are as likely women to develop it and initial disease activity is in the spinal cord and not in the brain. Primary Progressive MS often migrates into the brain, but is less likely to damage brain areas than relapsing/remitting or secondary progressive—for example, people with Primary Progressive are less likely to develop cognitive problems.

All forms of the disease, sites of CNS lesions and types of resulting symptoms or disorders are intended to be included within the scope of the present invention.

### Treatment of Multiple Sclerosis

Until the early 1990's, there was no significant treatment that could alter the course of the disease in the long term. Steroid therapy, although effective in shortening the duration of attacks, was never proven to affect the ultimate outcome of the disease nor consequent disability, and was therefore considered a symptomatic treatment of exacerbation. The last decade has witnessed the development of disease modifying treatments and improvement in symptomatic treatments. Three interferon-beta (IFN-β) products (Betaseron®, Avonex®, and Rebif®), which are immunomodulators, and two unrelated products (Copaxone® and Novantrone®), have been approved by the FDA.

All beta-interferons shut down the inflammation of MS lesions through various mechanisms including repairing the blood brain barrier and reducing the inflammatory process in the lesions. They are approved for relapsing-remitting MS. Depending upon the source, IFN-β is administered at different doses, following various time schedules, ranging from daily to weekly, by means of subcutaneous or intramuscular injections.

Long Term studies show that for most people beta interferon continue to be effective with continued use. However, as with other protein-based therapies, a sizeable portion of patients develop neutralizing antibodies (NAbs) to the drugs which reduce their efficacy. Twenty-eight to forty-seven percent of patients develop NAbs to IFN-β-1b and depending on the commercial source two to twenty-eight percent of patients develop NAbs to IFN-1-1a. The principle side effects of beta interferon are “flu-like symptoms” which can be very unpleasant and mild liver toxicity in some patients. More common problems are injection site reactions and the difficulties many people have injecting themselves.

Copaxone® (glatiramer acetate) is different from beta interferon in chemical structure and mechanisms of action. It consists of a group of synthetic polypeptides that looks something like myelin itself. It decreases the frequency and severity of attacks to the same extent as Betaseron® and Rebif®, but with slightly less effect on lesions as seen on Magnetic Resonance Imaging (MRI). Copaxone®, generally better tolerated than IFN-β products, is administered daily by subcutaneous injection and is used for relapsing-remitting MS. Due to the route of administration, the most common problem reported by patients is injection site reaction. Additional side effects reported thus far are mild and include transient flushing, chest and joint pains, weakness, nausea, anxiety and muscle stiffness. All above drugs are indicated for the treatment of a single form of the disease: relapsing-remitting MS.

Novantrone® (mitoxantrone) is a chemotherapy agent that slows disease progression in MS and lessens the number of relapses through its ability to suppress the activity of T cells and B cells. As opposed to previous drugs, Novantrone® is an immunosuppressor. It is approved for worsening MS including secondary progressive and relapsing-remitting forms of the disease and is considered a rescue therapy in patients whose disease is not controlled by beta interferon or glatiramer acetate. Novantrone(t is typically administered intravenously once every three months for a limited period not exceeding two years, due to its more severe side effects, cardiotoxicity and potential leukaemogenicity.

It should be appreciated that the orally effective cannabinoids of the invention overcome drawbacks of some or all of the existing DMAs in at least five aspects: (i) being non-protein based small molecules they have lower cost of production; (ii) they are less immunogenic and thus the risk of NAbs development is reduced; (iii) absence of NAbs ensure longer therapeutic efficacy over the years; (iv) oral administration increases patient compliance and eliminate injection site reactions; and (v) being CB₂ selective, compounds of the invention exhibit reduced side effects and toxicity.

As previously detailed MS can produce a wide range of symptoms which can be classified as visual, motor, sensory, coordination and balance, bowel, bladder, sexual, cognitive and others. The DMAs address the immune cause of the disease, whereas treatments including the administration of steroids, anti-convulsants, tricyclic antidepressants, anti-inflammatory drugs, non-steroidal anti-inflammatory drugs (NSAID), selective serotonin reuptake inhibitors (SSRI), monoamine oxidase inhibitors (MOI), antidepressants, benzodiazepines (BZD), muscle relaxants, anticholinergic agents, beta blockers, laxatives, and some specific channel blockers, target the symptoms. Such agents are used for the treatment of similar symptoms in diseases and disorders other than multiple sclerosis.

Steroids are described as one example of symptomatic treatments of MS and were the treatment of choice before the development of the DMAs. For acute exacerbations, steroids have been reported to shorten the duration of acute attacks by lessening the swelling and inflammation in MS lesions. However they do not alter the frequency of exacerbations or the progression of the MS, and long term use should be avoided except in selected patients. Included are synthetic adrenal glucocorticoids (corticosteroids) such as prednisone, prednisolone, methylprednisolone, betamethasone, and dexamethasone. Cortisones have an immunosuppressive effect and are believed to reduce the “leakiness” of the blood brain barrier. Steroids are merely palliative and do not address the cause of the disease. Some of the potentially severe side effects prevent prolonged use of steroids in the treatment of MS.

Other classes of compounds used in the prevention, alleviation or treatment of MS associated symptoms also suffer from serious side effects well known to the medical practitioner.

It should be appreciated that the orally effective cannabinoids of the invention may advantageously replace existing symptomatic treatment in view of previously reported and presently disclosed efficacy as: (i) anti-inflammatory agents; (ii) immunomodulating agents, (iii) analgesic agents; (iv) neuroprotective agents; (v) anti-oxidative agents; (v) anti-spasticity agents; and (vi) anti-tremor agents. Therefore, in view of their multiple activities the orally effective cannabinoids of the invention may be considered as multifactorial therapy, a fact that may further reduce the need for multiple drug treatment. Decreasing the absolute number of drugs necessary to treat MS, by eliminating the redundant agents, or reducing dosage of administration of some or all of the agents by coadministrating or combining the orally effective cannabinoids of the invention together with existing MS treatments, will decrease drug to drug interaction, and the overall side effects.

It will be apparent to persons skilled in the art that the ability of compounds of the invention to prevent symptoms associated with multiple sclerosis have a wider therapeutic benefice, since the same symptoms could be treated when occurring in other diseases or disorders. The treatment of MS associated symptoms, whether caused by multiple sclerosis or by other diseases or disorders, is encompassed within the scope of the present invention.

For example, neuropathic pain is not only observed in MS patients, but also in individuals suffering for instance from back pain, diabetes, cancer, monoradiculopathies, trigeminal neuralgia, postherpetic neuralgia, phantom limb pain, complex regional pain syndromes and the various peripheral neuropathies, or in patients receiving certain anti-neoplastic therapies. For instance, about 30% of patients receiving Taxol® therapy develop neuropathic pain. As in the case of MS, the existing therapies of neuropathic pain are considered unsatisfactory.

### Chemical Definitions

Some of the compounds according to the invention may exist in stereoisomeric forms which either are related as image and mirror image (enantiomers) or are not related as image and mirror image (diastereomers). The invention relates to the enantiomers or diastereomers or respective mixtures thereof. These mixtures of enantiomers and diastereomers can be separated into stereoisomerically uniform components in a known manner or synthesized a priori as separate enantiomers.

In the present invention we will refer to the following numbering of positions in the ring structure, where positions 1, 4 and 5 are chiral centers. The stereochemistry of the preferred (+)-α-pinene derivative the present invention is such that C-4 is S, the protons at C-1 and C-5 are cis in relation to one another and the protons at C-4 and C-5 are trans as shown in formula (II):

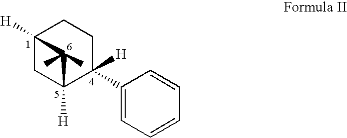

This nomenclature is equivalent to the alternative and previous definition of the stereochemistry which referred to chiral carbon C-5 instead of C-4. Namely, compounds of formula (II) can also be described as having a stereochemistry wherein C-5 is S, the protons at C-1 and C-5 are cis in relation to one another and the protons at C-4 and C-5 are trans.

In the present specification and claims which follow, certain compounds of the invention may be referred to by capital letters rather than by their full chemical names. For example, (−)-4-{4-[1,1-Dimethylheptyl]-2-fumarate-6-hydroxy-phenyl}-6,6-dimethyl-bicyclo[3.1.1]heptan-2-one is often referred to as compound 18F.

The alkyl substituents can be saturated or unsaturated, linear, branched or cyclic, the latter only when the number of carbon atoms in the alkyl chain is greater than or equal to three, and can contain mixed structures. When unsaturated, the hydrocarbon radicals may have one double bond, or more, and form alkenyls, or one triple bond, or more, and form alkynyls, all of which can be linear, branched or cyclic.

OC(O)R represents esters, OC(O)NR carbamates, OC(S)R thioesters, NR₂ amines, NRC(O)R amides, NRC(O)NR ureas, NRC(S)R thioamides, SR thiols or sulfides, S(O)R sulfoxides, SC(O)R thioesters, SC(O)NR thiocarbamates, SC(S)R dithioesters, S(O)(O)R sulfones, S(O)(O)NR sulfonamides, S(O)(O)NC(O)R acylsulfonamides, S(O)(O)NC(O)NR sulfonurea, S(O)(O)NC(S)R thioacylsulfonamide, P(O)(OR)₂ phosphate, OP(O)(OR)₂ ester phosphate, when R is a hydrogen or an alkyl chain.

“Halogen” or “halo” means fluorine (—F), chlorine (—Cl), bromine (—Br) or iodine (—I) and if more than one halogen is referred to (e.g., two or more variable groups may be a halogen), each halogen is independently selected.

The term “substituted” or “optionally substituted” means that one or more hydrogens on the designated atom is replaced or optionally replaced with a selection from the indicated group, provided that the designated atom's normal valency under the existing circumstances is not exceeded. Combination of substituents and/or variables are permissible only if such combinations result in stable compounds. By “stable compound” or “stable structure” is meant a compound that is sufficiently robust to survive isolation to a useful degree of purity from a reaction mixture, and formulation into an efficacious therapeutic agent.

Certain compounds of the invention are capable of further forming pharmaceutically acceptable salts and esters. “Pharmaceutically acceptable salts and esters” means any salt and ester that is pharmaceutically acceptable and has the desired pharmacological properties. Such salts, formed for instance by any carboxy or sulfo groups present in the molecule, include salts that may be derived from an inorganic or organic acid, or an inorganic or organic base, including amino acids, which is not toxic or otherwise unacceptable.

The present invention also includes within its scope solvates of compounds of formula (I) and salts thereof. “Solvate” means a physical association of a compound of the invention with one or more solvent molecules. This physical association involves varying degrees of ionic and covalent bonding, including hydrogen bonding. In certain instances the solvate will be capable of isolation. “Solvate” encompasses both solution-phase and isolatable solvates. Non-limiting examples of suitable solvates include ethanolates, methanolates and the like. “Hydrate” is a solvate wherein the solvent molecule is water.

In the present specification the term “prodrug” represents compounds which are rapidly transformed in vivo to parent compound of formula (I), for example by hydrolysis in the blood. Prodrugs are often useful because in some instances they may be easier to administer than the parent drug. They may, for instance, be bioavailable by oral administration whereas the parent drug is not. The prodrug may also have improved solubility compared to the parent drug in pharmaceutical compositions. All of these pharmaceutical forms are intended to be included within the scope of the present invention.

Pharmaceutically acceptable acid addition salts of the compounds include salts derived from inorganic acids such as hydrochloric, nitric, phosphoric, sulfuric, hydrobromic, hydriodic, phosphorous, and the like, as well as salts derived from organic acids such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Also contemplated are salts of amino acids such as arginate and the like and gluconate or galacturonate [Berge S. M. et al., J. of Pharmaceutical Science, 66: 1-19, 1977].

The acid addition salts of said basic compounds are prepared by contacting the free base form with a sufficient amount of the desired acid to produce the salt in the conventional manner. The free base form may be regenerated by contacting the salt form with a base and isolating the free base in the conventional manner. The free base forms differ from their respective salt forms somewhat in certain physical properties such as solubility in polar solvents, but otherwise the salts are equivalent to their respective free base for purposes of the present invention.

The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention.

Certain compounds used in the methods of the present invention are known and were disclosed in International patent application WO 03/063758 which claims novel (+) α-pinene derivatives that exhibit selectivity for the CB₂ receptor. This application disclosed that hydrophobic compounds of the invention are useful in the treatment of MS when administered intravenously, and suggested that certain compounds might be delivered orally, oral efficacy of water-soluble (+) α-pinene derivatives was not specifically demonstrated.

It was previously shown that pretreatment with compound 18F of the invention, also known as PRS-211,375, was effective against acute pain when administered orally [Bar-Joseph A. et al., Society For Neuroscience: Program No. 909.5, 2003]. However, these experiments did not teach that said compound could be effective for chronic administration to treat an established disease such as multiple sclerosis. None of the other compounds of the invention were previously shown to be effective in vivo after oral administration.

### Pharmacology

In the present specification and claims which follow the compositions comprising an orally effective compound are intended to encompass both prophylactically and therapeutically effective compositions.

The term “prophylactically effective” is intended to qualify the amount of compound which will achieve the goal of prevention, reduction or eradication of the risk of occurrence of the disorder, while avoiding adverse side effects. The term “therapeutically effective” is intended to qualify the amount of compound that will achieve, with no adverse effects, alleviation, diminished progression or treatment of the disorder, once the disorder cannot be further delayed and the patients are no longer asymptomatic, hence providing either a subjective relief of a symptom (s) or an objectively identifiable improvement as noted by the clinician or other qualified observer.

Insidious neurological progression suggestive of MS can be detected at pre-symptomatic stage of the disease, especially in individuals at risk due to family history. MS is known to be associated with genetic predisposition, though the exact genes involved are not yet characterized. The tests used for diagnosis of MS include Magnetic Resonance Imaging (MRI); Computed Tomography (CT) scans; Lumbar puncture and analysis of cerebrospinal fluid (CSF); and finally Evoked Potential (EP) tests which can be subdivided into Visually Evoked Potential (VEP), Brainstem Auditory Evoked Response (BAER) and SomatoSensory Evoked Potential (SSEP). Identification of presymptomatic individuals at risk allows the prophylactic administration of the compositions of the invention to prevent the overt onset of the disease.

The “individual” or “patient” for purposes of treatment includes any human or animal affected by any of the diseases where the treatment has beneficial therapeutic impact. Usually, the animal that serves to establish the pre-clinical data and that can be treated by compounds of the invention is a vertebrate such as a primate including chimpanzees, monkeys and macaques, a rodent including mice, rats, ferrets, rabbits and hamsters, a domestic or game animal including bovine species, equine species, pigs, sheeps, caprine species, feline species, canine species, avian species, and fishes

Hereinafter, the term “oral administration” includes, but is not limited to, administration by mouth for absorption through the gastrointestinal tract (peroral) wherein the drug is swallowed, or for trans-mucosal absorption in the oral cavity by buccal, gingival, lingual, sublingual and oro-pharyngeal administration. Compositions for oral administration include powders or granules, suspensions or solutions in water or non-aqueous media, sachets, capsules or tablets. Thickeners, diluents, flavorings, dispersing aids, emulsifiers, binders or preservatives may be desirable.

It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

The pharmaceutical compositions may contain in addition to the active ingredient conventional pharmaceutically acceptable carriers, diluents and excipients necessary to produce a physiologically acceptable and stable formulation. The terms carrier, diluent or excipient mean an ingredient that is compatible with the other ingredients of the compositions disclosed herein, especially substances which do not react with the compounds of the invention and are not overly deleterious to the patient or animal to which the formulation is to be administered. Enabling therapeutically effective and convenient administration of the compounds of the present invention is an integral part of this invention.

The pharmaceutical compositions may be in a liquid, aerosol or solid dosage form, and may be formulated into any suitable formulation including, but not limited to, solutions, suspensions, micelles, emulsions, microemulsions, aerosols, capsules, tablets, and the like, as will be required for the oral route of administration.

Solid compositions for oral administration such as tablets, pills, capsules, softgels or the like may be prepared by mixing the active ingredient with conventional, pharmaceutically acceptable ingredients such as corn starch, lactose, sucrose, mannitol, sorbitol, talc, polyvinylpyrrolidone, polyethyleneglycol, cyclodextrins, dextrans, glycerol, polyglycolized glycerides, tocopheryl polyethyleneglycol succinate, sodium lauryl sulfate, polyethoxylated castor oils, non-ionic surfactants, stearic acid, magnesium stearate, dicalcium phosphate and gums as pharmaceutically acceptable diluents. The tablets or pills can be coated or otherwise compounded with pharmaceutically acceptable materials known in the art, such as microcrystalline cellulose and cellulose derivatives such as hydroxypropylmethylcellulose (HPMC), to provide a dosage form affording prolonged action or sustained release. Liquid forms may be prepared for oral administration The liquid compositions include aqueous solutions, with or without organic cosolvents, aqueous or oil suspensions including but not limited to cyclodextrins as suspending agent, flavored emulsions with edible oils, triglycerides and phospholipids, as well as elixirs and similar pharmaceutical vehicles. In addition, the compositions of the present invention may be formed as aerosols, for buccal and oropharyngeal administration. The aerosol is conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount.

Pharmaceutical compositions of the present invention may be manufactured by processes well known in the art, e.g., by means of conventional mixing, dissolving, granulating, grinding, pulverizing, dragee-making, levigating, emulsifying, encapsulating, entrapping or lyophilizing processes.

Prior to their use as medicaments, the pharmaceutical compositions will generally be formulated in unit dosage. The active dose for humans can be determined by standard clinical techniques and is generally in the range of from 0.01 mg to about 50 mg per kg body weight, in a regimen of 1-4 times a day. The preferred range of dosage varies with the specific compound used and is generally in the range of from 0.1 mg to about 20 mg per kg body weight. However, it is evident to one skilled in the art that dosages would be determined by the attending physician, according to the disease or disorder to be treated, its severity, the method and frequency of administration, the patient's age, weight, gender and medical condition, concurrent treatment, if any, contraindications and the like.

Effective doses may be extrapolated from dose-response curves derived from in vitro or animal model test systems. For example, in order to obtain an estimated effective mg/kg dose for humans based on data generated from mice or rat studies, the effective mg/kg dosage in mice or rats is divided by twelve or six, respectively.

Pharmaceutical compositions of the present invention may also include one or more additional active ingredients. In particular, the orally active cannabinoids of the invention may be coadministered or used in combination with one or more other drugs used in the treatment of MS.

The second MS treating agents, which can be the same or different from each other, are independently selected form the group consisting of immunomodulators, IFN-β, IFN-β-1a, IFN-β-1b, glatiramer acetate, immunosuppressor, azathioprine, cladribine, cyclophosphamide, mitoxantrone, steroids, anti-convulsants, tricyclic antidepressants, anti-inflammatory drugs, non-steroidal anti-inflammatory drugs (NSAID), selective serotonin reuptake inhibitors (SSRI), monoamine oxidase inhibitors (MOI), antidepressants, benzodiazepines (BZD), muscle relaxants, anticholinergic agents, beta blockers, laxatives, and some specific channel blockers.

In a further aspect, the invention provides a method for alleviating or treating multiple sclerosis comprising the step of administering to a patient in need thereof at least one compound of formula (I) in combination with at least one compound selected from the group consisting of Avonex®, Betaseron®, Rebif®, Copaxone®, Novantrone® and other compounds indicated for the treatment of multiple sclerosis.

The administration and dosage of such second agents is according to the schedule listed in the product information sheet of the approved agents, in the Physicians Desk Reference (PDR) as well as therapeutic protocols well known in the art.

When two or more active ingredients are administered to achieve the therapeutic goals of the present invention, co-administration can be in a unique dosage form for or in separate dosage forms for combined administration. Combined administration in the context of this invention is defined to mean the administration of more than one therapeutic in the course of a coordinated treatment to achieve an improved clinical outcome. Such combined administration may occur at the same time and also be coextensive, that is, occurring during overlapping periods of time.

A further aspect of the present invention provides a method of preventing, alleviating or treating multiple sclerosis, comprising the step of administering to a patient in need thereof an orally effective amount of a pharmaceutical composition comprising as an active ingredient a compound of formula (I):

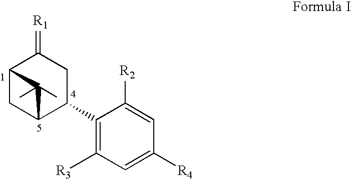

having a specific stereochemistry wherein C-4 is S, the protons at C-1 and C-5 are cis in relation to one another and the protons at C-4 and C-5 are trans; and wherein:
R₁ is selected from the group consisting of
(a) O or S,
(b) C(R′)₂ wherein R′ at each occurrence is independently selected from the group consisting of hydrogen, cyano, —OR″, —N(R″)₂, a saturated or unsaturated, linear or branched C₁-C₆ alkyl, C₁-C₆ alkyl-OR″ or C₁-C₆ alkyl-N(R″)₂ wherein at each occurrence R″ is independently selected from the group consisting of hydrogen, C(O)R′″, C(O)N(R′″)₂, C(S)R′″, saturated or unsaturated, linear or branched C₁-C₆ alkyl, C₁-C₆ alkyl-OR′″, and C₁-C₆ alkyl-N(R′″)₂, wherein at each occurrence R′″ is independently selected from the group consisting of hydrogen or saturated or unsaturated, linear, branched or cyclic C₁-C₁₂ alkyl, and
(c) NR″ or N—OR″ wherein R″ is as previously defined;
   R₂ and R₃ are each independently selected from the group consisting of
(a) —R″, —OR″, —N(R″)₂, —SR″, —S(O)(O)NR″, wherein at each occurrence R″ is as previously defined,
(b) —S(O)R^{b}, —S(O)(O)R^{b} wherein R^{b} is selected from the group consisting of hydrogen, saturated or unsaturated, linear or branched C₁-C₆ alkyl, C₁-C₆ alkyl-OR″, and C₁-C₆ alkyl-N(R″)₂, wherein R″ is as previously defined, and
(c) —OC(O)OH, —OS(O)(O)OR^{e}, —OP(O)(OR^{e})₂, —OR^{d} or —OC(O)—R^{d} chain terminated by —C(O)OH, —S(O)(O)OR^{e}, or —P(O)(OR^{e})₂, wherein R^{d} is a saturated or unsaturated, linear or branched C₁-C₆ alkyl and R^{e} is at each occurrence selected from the group consisting of hydrogen and R^{d} as previously defined; and
   R₄ is selected from the group consisting of
(a) R wherein R is selected from the group consisting of hydrogen, halogen, OR′″, OC(O)R′″, C(O)OR′″, C(O)R′″, OC(O)OR′″, CN, N(R′″)₂, NC(O)R′″, NC(O)OR′″, C(O)N(R′″)₂, NC(O)N(R′″)₂, and SR′″, wherein at each occurrence R′″ is as previously defined,
(b) a saturated or unsaturated, linear, branched or cyclic C₁-C₁₂ alkyl-R wherein R is as previously defined,
(c) an aromatic ring which can be further substituted at any position by R wherein R is as previously defined, and
(d) a saturated or unsaturated, linear, branched or cyclic C₁-C₁₂ alkyl optionally terminated by an aromatic ring which can be further substituted as defined in (c);
and pharmaceutically acceptable salts, esters or solvates thereof.

According to certain embodiments, the present invention provides a method of preventing, alleviating or treating multiple sclerosis, comprising the step of administering to an individual in need thereof an orally effective amount of a pharmaceutical composition comprising as an active ingredient a compound of formula (I) wherein R₁ is O, R₂ and R₃ are each OR^{f} wherein at each occurrence R^{f} is independently selected from the group consisting of hydrogen, —R^{d} and —C(O)—R^{d}, wherein R^{d} is a saturated or unsaturated, linear or branched C₁-C₆ alkyl chain terminated by —C(O)OR^{g} and R^{g} is hydrogen or a saturated or unsaturated, linear or branched C₁-C₆ alkyl, and R₄ is selected from the group consisting of a saturated or unsaturated, linear, branched or cyclic C₁-C₁₂ alkyl-R^{h} wherein R^{h} is selected from the group consisting of R and an aromatic ring which can be optionally further substituted at any position by R as previously defined.

According to additional embodiments, the present invention provides a method of preventing, alleviating or treating multiple sclerosis, comprising the step of administering to a patient in need thereof an orally effective amount of a pharmaceutical composition comprising as an active ingredient a compound of formula (I) wherein R₁ is O, R₂ and R₃ are each independently selected from the group consisting of OH, succinate, fumarate, and methylenoxycarboxyl, and R₄ is selected from the group consisting of 1,1-dimethylpentyl, 1,1-dimethylheptyl, 1,1-dimethyl-6-heptynyl, 1,1-dimethyl-3-phenyl-propyl, 1,1,3-trimethyl-butyl, 1-(4-chloro-phenyl)-1-methyl-ethyl, 1-ethyl-1-methyl-propyl, 5-bromo-1,1-dimethyl pentyl or 1,1-dimethyl-pent-4-enyl.

According to an exemplary embodiment, the present invention provides a method of preventing, alleviating or treating multiple sclerosis, comprising the step of administering to a patient in need thereof an orally effective amount of a pharmaceutical composition comprising as an active ingredient a compound of formula (I) wherein R₁ is O, R₂ is OH, R₃ is fumarate and R₄ is 1,1-dimethylheptyl.

According to another aspect, the present invention provides a method of preventing, alleviating or treating neurological symptoms selected from the list consisting of tremor, spasticity, muscle weakness, and lack of coordination, comprising the step of administering to an individual in need thereof an orally effective amount of a pharmaceutical composition comprising as an active ingredient a compound of formula (I) as defined above.

According to another aspect, the present invention provides a method of modulating mediators of inflammation comprising the step of administering to an individual in need thereof an orally effective amount of a pharmaceutical composition comprising as an active ingredient a compound of formula (I) as defined above.

A further aspect of the present invention provides the use for the preparation of a medicament for preventing, alleviating or treating multiple sclerosis, of an orally effective compound of formula (I):

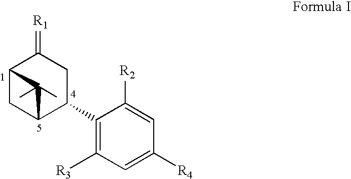

having a specific stereochemistry wherein C-4 is S, the protons at C-1 and C-5 are cis in relation to one another and the protons at C-4 and C-5 are trans; and wherein:
R₁ is selected from the group consisting of
(a) O or S,
(b) C(R′)₂ wherein R′ at each occurrence is independently selected from the group consisting of hydrogen, cyano, —OR″, —N(R″)₂, a saturated or unsaturated, linear or branched C₁-C₆ alkyl, C₁-C₆ alkyl-OR″ or C₁-C₆ alkyl-N(R″)₂ wherein at each occurrence R″ is independently selected from the group consisting of hydrogen, C(O)R′″, C(O)N(R′″)₂, C(S)R′″, saturated or unsaturated, linear or branched C₁-C₆ alkyl, C₁-C₆ alkyl-OR′″, and C₁-C₆ alkyl-N(R′″)₂, wherein at each occurrence R′″ is independently selected from the group consisting of hydrogen or saturated or unsaturated, linear, branched or cyclic C₁-C₁₂ alkyl, and
(c) NR″ or N—OR″ wherein R″ is as previously defined;
   R₂ and R₃ are each independently selected from the group consisting of
(a) —R″, —OR″, —N(R″)₂, —SR″, —S(O)(O)NR″, wherein at each occurrence R″ is as previously defined,
(b) —S(O)R^{b}, —S(O)(O)R^{b} wherein R^{b} is selected from the group consisting of hydrogen, saturated or unsaturated, linear or branched C₁-C₆ alkyl, C₁-C₆ alkyl-OR″, and C₁-C₆ alkyl-N(R″)₂, wherein R″ is as previously defined, and
(c) —OC(O)OH, —OS(O)(O)OR^{c}, —OP(O)(OR^{c})₂, —OR^{d} or —OC(O)—R^{d} chain terminated by —C(O)OH, —S(O)(O)OR^{c}, or —P(O)(OR^{e})₂, wherein R^{d} is a saturated or unsaturated, linear or branched C₁-C₆ alkyl and R^{c} is at each occurrence selected from the group consisting of hydrogen and R^{d} as previously defined; and
   R₄ is selected from the group consisting of
(a) R wherein R is selected from the group consisting of hydrogen, halogen, OR′″, OC(O)R′″, C(O)OR′″, C(O)R′″, OC(O)OR′″, CN, N(R′″)₂, NC(O)R′″, NC(O)OR′″, C(O)N(R′″)₂, NC(O)N(R′″)₂, and SR′″, wherein at each occurrence R′″ is as previously defined,
(b) a saturated or unsaturated, linear, branched or cyclic C₁-C₁₂ alkyl-R wherein R is as previously defined,
(c) an aromatic ring which can be further substituted at any position by R wherein R is as previously defined, and
(d) a saturated or unsaturated, linear, branched or cyclic C₁-C₁₂ alkyl optionally terminated by an aromatic ring which can be further substituted as defined in (c);
and pharmaceutically acceptable salts, esters or solvates thereof.

According to certain embodiments, the present invention provides the use for the preparation of a medicament for preventing, alleviating or treating multiple sclerosis, of an orally effective compound of formula (I) wherein R₁ is O, R₂ and R₃ are each OR^{f} wherein at each occurrence R^{f} is independently selected from the group consisting of hydrogen, —R^{d} and —C(O)—R^{d}, wherein R is a saturated or unsaturated, linear or branched C₁-C₆ alkyl chain terminated by —C(O)OR^{g} and R^{g} is hydrogen or a saturated or unsaturated, linear or branched C₁-C₆ alkyl, and R₄ is selected from the group consisting of a saturated or unsaturated, linear, branched or cyclic C₁-C₁₂ alkyl-R^{h} wherein R^{h} is selected from the group consisting of R and an aromatic ring which can be optionally further substituted at any position by R as previously defined.

According to additional embodiments, the present invention provides the use for the preparation of a medicament for preventing, alleviating or treating multiple sclerosis, of an orally effective compound of formula (I) wherein R₁ is O, R₂ and R₃ are each independently selected from the group consisting of OH, succinate, fumarate, and methylenoxycarboxyl, and R₄ is selected from the group consisting of 1,1-dimethylpentyl, 1,1-dimethylheptyl, 1,1-dimethyl-6-heptynyl, 1,1-dimethyl-3-phenyl-propyl, 1,1,3-trimethyl-butyl, 1-(4-chloro-phenyl)-1-methyl-ethyl, 1-ethyl-1-methyl-propyl, 5-bromo-1,1-dimethyl pentyl or 1,1-dimethyl-pent-4-enyl.

According to an exemplary embodiment, the present invention provides the use for the preparation of a medicament for preventing, alleviating or treating multiple sclerosis, of an orally effective compound of formula (I) wherein R₁ is O, R₂ is OH, R₃ is fumarate and R₄ is 1,1-dimethylheptyl.

According to another aspect, the present invention provides the use for the preparation of a medicament for preventing, alleviating or treating neurological symptoms as previously defined, of an orally effective compound of formula (I) as defined above.

According to another aspect, the present invention provides the use for the preparation of a medicament for modulating mediators of inflammation of an orally effective compound of formula (I) as defined above.

The principles of the present invention will be more fully understood by reference to the following examples, which illustrate preferred embodiments of the invention and are to be construed in a non-limitative manner.

### EXAMPLES

The following examples are provided in order to demonstrate and further illustrate certain preferred embodiments and aspects of the present invention and are not to be construed as limiting the scope thereof. Most of the techniques used to prepare the in vitro or in vivo models, testing the compounds and analyzing the outcome are widely practiced in the art, and most practitioners are familiar with the standard resource materials that describe specific conditions and procedures. However, for convenience, the following descriptions may serve as guidelines.

In the experimental disclosure which follows, the following abbreviations apply: N (normal); M (molar); mM (millimolar); μM (micromolar); mmol (millimole); kg (kilograms); g (grams); mg (milligrams); μg (micrograms); ng (nanograms); pg (picograms); ml (milliliters); μl (microliters); mm (millimeters); μm (micrometers); h (hours); min (minutes); ° C. (degrees Centigrade); i.p. (intraperitoneally); i.v. (intraperitoneously); p.o. (per os); s.c. (subcutaneously); AUC (Area Under the Curve); SD (standard deviation); SEM (standard error of the mean); nr (not relevant) and ns (not significant).

For convenience and better understanding, the section of the Examples is divided into two subsections: the Chemical Section describing the synthesis of compounds of the invention, some of their physicochemical properties and their formulation, and the Biological Section describing the biological activity of the compounds.

### Chemical Section

The synthesis of some compounds of the invention was previously disclosed in International Patent Application WO 03/063758. These processes are reproduced hereinbelow for convenience. Compounds 18A to 18F were previously disclosed as compounds A, R, S, T, Y and Z, respectively in WO 03/063758. Compounds 18G to 18I are novel derivatives using similar starting material or synthetic schemes. It is clear to person skilled in the art of synthesis of cannabinoid compounds that alternative synthetic processes exist.

### Example 1

### Synthesis of Compound 18A: (−)-4-[4-(1,1-Dimethylheptyl)-2,6-dihydroxy-phenyl]-6,6-dimethyl-bicyclo[3.1.1]heptan-2-one

The synthesis of compound 18A is depicted in Scheme 1 when the R moiety of the resorcinol compound is 1,1-dimethylheptyl.

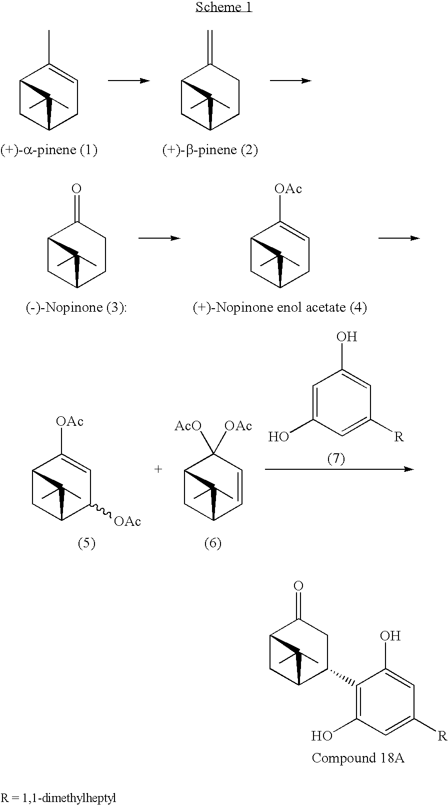

To a 3-necked flask containing n-butyl lithium (196 ml, 2M) and 44 g potassium tert-butoxide at −78° C. under nitrogen atmosphere, 50 ml of (+)-α-pinene (1) was added dropwise. The reaction was allowed to warm up to room temperature and was stirred continuously for 48 hours. The reaction was then cooled to −78° C. Trimethyl borate (113 ml) in 80 ml of ether was added and the reaction was allowed to warm up to room temperature and was stirred for one additional hour. The organic layer was separated, and the aqueous layer was extracted with n-hexane (3×80 ml). The combined organic phases were washed with brine, dried over anhydrous sodium sulfate, filtered and evaporated to dryness to afford compound (2), (+)-β-pinene. This procedure is according to Brown et al. [Brown H. C. et al., J. Org. Chem. 54: 1764-6, 1989]. To (+)-β-pinene (2) (30.8 g) were added RuCl₃ (0.470 g), and benzyltributyl ammonium chloride (2.12 g) dissolved in 250 ml of ethyl acetate. To this mixture, sodium periodate (145.5 g) in 1.3 L of water was added dropwise, stirred at room temperature for 3 hours and left overnight. 250 ml of ethyl acetate were added to the reaction mixture. The organic phase was separated, washed with 500 ml of brine, 500 ml of 10% sodium sulfite, dried over anhydrous sodium sulfate, filtered, evaporated under reduced pressure to afford compound (3), (−)-Nopinone. This procedure is according to Yuasa et al. [Yuasa Y. et al., J. Essent. Oil. Res. 10: 39-42, 1998]. (−)-Nopinone (3) (14.86 g) and p-toluenesulfonic acid (1.48 g) were dissolved in isoprenyl acetate (148 ml). The reaction mixture was heated at reflux for 5 hours using a Dean-Stark apparatus to remove the acetone. The solvents were removed under reduced pressure, and the residue was taken in 400 ml of ether, washed with water, dried over anhydrous sodium sulfate, filtered and evaporated to afford compound (4), (+)-Nopinone enol acetate. This procedure is based on a method developed for the opposite enantiomer by Archer et al. [Archer R. A. et al., J. Org. Chem. 42: 2277-84, 1977]. To a solution of 16.17 g of (+)-Nopinone enol acetate (4) in 202 ml of dry toluene were added 62.2 g of Pb(OAc)₄ (previously dried in vacuo over P₂O₅/KOH overnight). The reaction mixture was heated at 80° C. for 3.5 hours, cooled, filtered, washed with saturated sodium bicarbonate. The organic layer was separated, dried over anhydrous sodium sulfate and evaporated under reduced pressure to yield (+)-6,6-Dimethyl-2,4-diacetoxy-2-norpinene (5) and (−)-6,6-dimethyl-2,2-diacetoxy-3-norpinene (6). A mixture of 5 and 6 (1.18 g, 5 mmol), resorcinol wherein R is 1,1-dimethylheptyl (7) (1.18 g, 5 mmol) and p-toluenesulfonic acid (0.95 g, 5 mmol) in chloroform (50 ml) was allowed to react at room temperature for 4 hours. Ether (30 ml) was then added, and the organic phase was washed with saturated sodium bicarbonate, water, then dried over anhydrous sodium sulfate, filtered and evaporated. The residue was allowed to crystallize in acetonitrile to provide 0.5 g of crystals. The mother liquors were chromatographed over silica gel to afford further 0.7 g of pure compound 18A.

### Example 2

### Synthesis of Compound 18B: (−)-4-{4-[1,1-dimethylheptyl]-2-succinate-6-hydroxy-phenyl}-6,6-dimethyl-bicyclo[3.1.1]heptan-2-one

The synthesis of compound 18B is depicted in Scheme 2.

### Example 3

### Synthesis of Compound 18C: (−)-4-{4-[1,1-dimethylheptyl]-2,6-disuccinate-phenyl}-6,6-dimethyl-bicyclo[3.1.1]heptan-2-one

The synthesis of compound 18C is depicted in Scheme 2.

A mixture of compound 18A (227 mg, 0.61 mmole) and succinic anhydride (731 mg, 7.31 mmole) in dry pyridine (10 ml) was heated to 50° C., under N₂ atmosphere. Potassium t-butoxide was added and the obtained mixture was stirred overnight (50° C.). The mixture was poured into 1 N HCl, and extracted with ethyl acetate. The combined organic phase was washed with 1 N HCl and brine, dried (Na₂SO₄) and evaporated. The two products were separated by column chromatography (20% ethyl acetate/petroleum ether+0.1% acetic acid) to yield 220 mg of compound 18B and 150 mg of compound 18C.

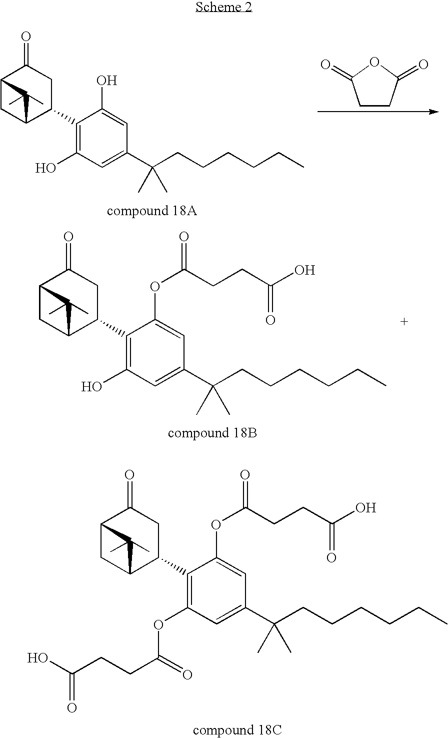

### Example 4

### Synthesis of Compound 18D: (−)-4-{4-[1,1-dimethylheptyl]-2,6-bi-diethylphosphate-phenyl}-6,6-dimethyl-bicyclo[3.1.1]heptan-2-one

The synthesis of compound 18D is depicted in Scheme 3.

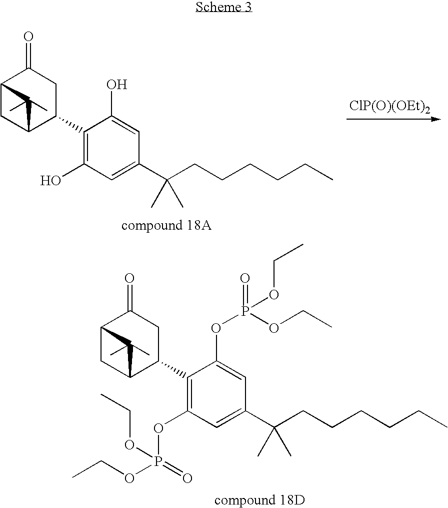

Reaction was carried out under N₂ atmosphere. To a well-stirred solution of compound 18A (1.97 g, 5.29 mmole) in freshly distilled THF, potassium t-butoxide (1.54 g, 13.75 mmole) was added and the mixture was stirred for 10 minutes. Diethyl chlorophosphate was added then and the reaction mixture was stirred overnight. Water was added and the aqueous phase was extracted with ethyl acetate. The combined organic layers were washed with brine, dried (Na₂SO₄) and evaporated. Purification by chromatography on silica-gel using 25%-70% ethyl acetate-petroleum ether as eluent gave 2.2 g of pure compound 18D.

### Example 5

### Synthesis of Compound 18E: (−)-4-{4-[1,1-Dimethylpentyl]-2-succinate-6-hydroxy-phenyl}-6,6-dimethyl-bicyclo[3.1.1]heptan-2-one

The synthesis of compound 18E is similar to the synthesis of compound 18B depicted in Scheme 2. The only difference resides in the starting material, while compound 18A yields compound 18B, the starting material of compound 18E is prepared using the same synthetic procedure as 18A when the R moiety of the resorcinol compound is 1,1-dimethylpentyl instead of 1,1-dimethylheptyl.

### Example 6

### Synthesis of Compound 18F: (−)-4-{4-[1,1-Dimethylheptyl]-2-fumarate-6-hydroxy-phenyl}-6,6-dimethyl-bicyclo[3.1.1]heptan-2-one

The synthesis of compound 18F is depicted in Scheme 4.

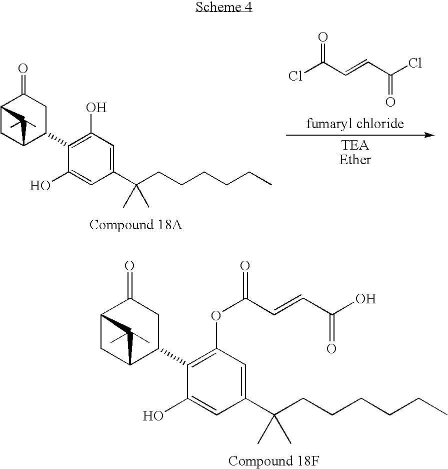

Compound 18A (600 mg, 1.6 mmol) was dissolved in 100 ml of dry diethyl ether. Then 0.21 ml of triethylamine (1.6 mmol) was added and 0.18 ml of fumaryl chloride (1.7 mmol). After stirring for about 15 minutes, the salt trimethylammonium chloride was filtered and the filtrate was evaporated. Then ethyl acetate was added to the residue and washed three times with water until the pH was above 4. The organic phase was then washed with saturated sodium chloride, dried over sodium sulfate, filtered and evaporated. Compound 18F was then purified by column chromatography on silica gel using 20% ethyl acetate and petroleum ether as eluent.

### Example 7

### Synthesis of Compound 18G: (−)-4-{4-[1,1-Dimethylheptyl]-2,6-difumarate-phenyl}-6,6-dimethyl-bicyclo[3.1.1]heptan-2-one

The synthesis of compound 18G is depicted in Scheme 5.

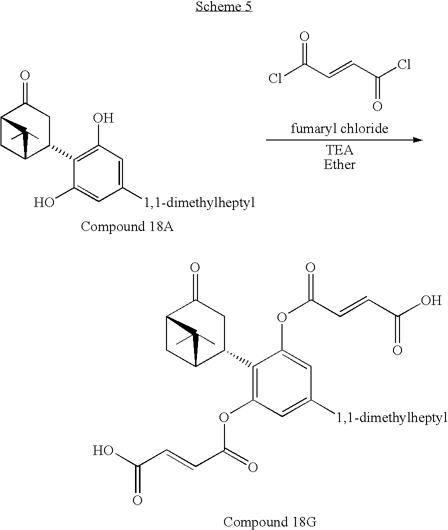

Compound 18A (2 g, 5.37 mmol) was dissolved in 100 ml of dry diethyl ether and cooled to −40° C. Then 1.9 ml of triethylamine (13.71 mmol) was added and 1.5 ml of fumaryl chloride (13.73 mmol). After stirring for about 15 minutes the mixture at 40° C., 100 ml of water were added and further stirred for 15 minutes at room temperature. Then the reaction mixture was transferred to a separating funnel and the aqueous layer is extracted three times with 300 ml each of ethyl acetate. The organic phase was then washed with water followed by saturated sodium chloride, dried over sodium sulfate, filtered and concentrated under reduced pressure. Compound 18G was then purified by flash chromatography on silica gel using petroleum ether and ethyl acetate in 10:90 ratio as eluent. 0.68 g of pure compound 18G was afforded (24% yield).

### Example 8

### Synthesis of Compound 18H: (−)-4-{4-[1,1-Dimethylpentyl]-2-fumarate-6-hydroxy-phenyl}-6,6-dimethyl-bicyclo[3.1.1]heptan-2-one

The synthesis of compound 18H is similar to the synthesis of compound 18F depicted in Scheme 4. The only difference resides in the starting material, while compound 18A yields compound 18F, the starting material of compound 18H is prepared using the same synthetic procedure as 18A when the R moiety of the resorcinol compound is 1,1-dimethylpentyl instead of 1,1-dimethylheptyl.

### Example 9

### Synthesis of Compound 18I: (−)-4-{4-[1,1-Dimethylheptyl]-2-(methylenoxycarboxyl)-6-hydroxy-phenyl}-6,6-dimethyl-bicyclo[3.1.1]heptan-2-one

The synthesis of compound 18I is depicted in Scheme 6.

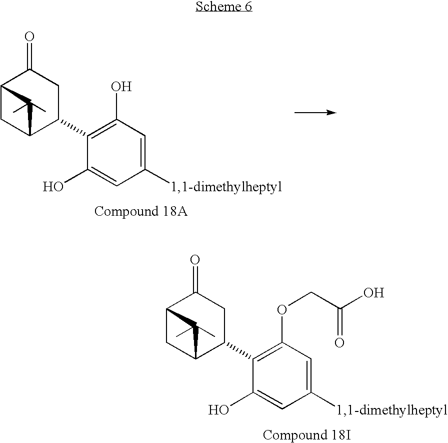

Compound 18A (374 mg, 1 mmol) was dissolved in 20 ml of dichloromethane at room temperature and 20 mg of copper bromide were added (solution A). In a separate vessel, to 10 ml dichloromethane were added 148 mg (1.01 mM) of diazotertbutylacetate. The second solution was added to solution A dropwise over a period of 5 minutes. The resulting reaction mixture was stirred for 3 hours at room temperature. Then the reaction mixture was filtered and the filtrate was concentrated to dryness under reduced pressure. The residue was dissolved in dichloromethane:trifluoroacetic acid TFA (10:1) and stirred for 2 hours at room temperature. The resulting solution was concentrated to dryness under reduced pressure and the residue was purified by column chromatography on silica gel using petroleum ether and ethyl acetate in 6:1 ratio as eluent to afford 7 mg pure compound 181.

### Example 10

### Physicochemical Properties

Following the discovery of the activities of compound 18A previously disclosed in International Patent Application WO 03/063758, efforts were made to synthesize related compounds that would be more water soluble or would hydrolyze in vivo to parent compound, compounds 18B to 18I being non limiting examples of this approach.

Compounds which bind preferably to CB₂ and have water solubility superior to that of Δ⁹-THC include: (−)-4-[4-(1,1-Dimethyl-hept-6-ynyl)-2,6-dihydroxy-phenyl]-6,6-dimethyl-bicyclo[3.1.1]heptan-2-one; (−)-4-[4-(1,1-Dimethyl-3-phenyl-propyl)-2,6-dihydroxy-phenyl]-6,6-dimethyl-bicyclo[3.1.1]heptan-2-one; (−)-4-[2,6-dihydroxy-4-(1,1,3-trimethyl-butyl)-phenyl]-6,6-dimethyl-bicyclo[3.1.1]heptan-2-one; (−)-4-{4-[1-(4-chloro-phenyl)-1-methyl-ethyl]-2,6-dihydroxy-phenyl]-6,6-dimethyl-bicyclo[3.1.1]heptan-2-one; (−)-4-[4-(1,1-Dimethyl-pentyl)-2,6-dihydroxy-phenyl]-6,6-dimethyl-bicyclo[3.1.1]heptan-2-one; (−)-4-[4-(1-ethyl-1-methyl-propyl)-2,6-dihydroxy-phenyl]-6,6-dimethyl-bicyclo[3.1.1]heptan-2-one; (−)-4-[4-(5-Bromo-1,1-Dimethylpentyl)-2,6-dihydroxy-phenyl]-6,6-dimethyl-bicyclo[3.1.1]heptan-2-one; (−)-4-{4-[1,1-dimethylheptyl]-2-succinate-6-hydroxy-phenyl}-6,6-dimethyl-bicyclo[3.1.1]heptan-2-one; (−)-4-{4-[1,1-dimethylheptyl]-2,6-disuccinate-phenyl}-6,6-dimethyl-bicyclo[3.1.1]heptan-2-one; (−)-4-[4-(1,1-Dimethyl-pent-4-enyl)-2,6-dihydroxy-phenyl]-6,6-dimethyl-bicyclo[3.1.1]heptan-2-one; (−)-4-{4-[1,1-Dimethylpentyl]-2-succinate-6-hydroxy-phenyl}-6,6-dimethyl-bicyclo[3.1.1]heptan-2-one; (−)-4-{4-[1,1-Dimethylheptyl]-2-fumarate-6-hydroxy-phenyl}-6,6-dimethyl-bicyclo[3.1.1]heptan-2-one; (−)-4-{4-[1,1-Dimethylheptyl]-2,6-difumarate-phenyl}-6,6-dimethyl-bicyclo[3.1.1]heptan-2-one; (−)-4-{4-[1,1-Dimethylpentyl]-2-fumarate-6-hydroxy-phenyl}-6,6-dimethyl-bicyclo[3.1.1]heptan-2-one; and (−)-4-{4-[1,1-Dimethylheptyl]-2-(methylenoxycarboxyl)-6-hydroxy-phenyl}-6,6-dimethyl-bicyclo[3.1.1]heptan-2-one.

Information regarding certain physicochemical properties of some of these compounds is presented in the following table. Expected water solubility, logP and logD at pH 7 were calculated using Advanced Chemistry Development software (ACD labs, version 4.04). THC and Nabilone, being both commercially available cannabinoids, are reported herein as references.

TABLE 1
Selected physicochemical properties

| | | | | | | LogD, |
|---|---|---|---|---|---|---|
| Compound | Structure | MW | Solubility | | LogP | pH7 |
| Δ⁹-THC | 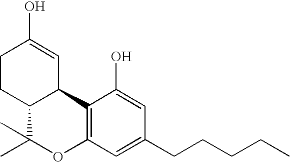 | 314.46 | 400 | ng/ml | 7.68 ± 0.35 | 7.68 |
| Nabilone | 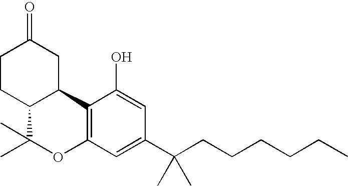 | 372.54 | 8 | ng/ml | 7.01 ± 0.39 | 7.01 |
| 18A | 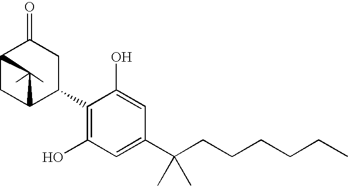 | 372.54 | 76 | ng/ml | 6.21 ± 0.38 | 6.21 |
| 18B | 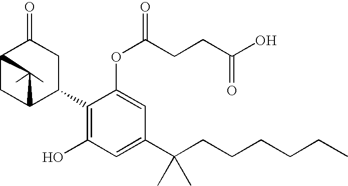 | 472.61 | 24 | μg/ml | 6.49 ± 0.40 | 3.76 |
| 18C | 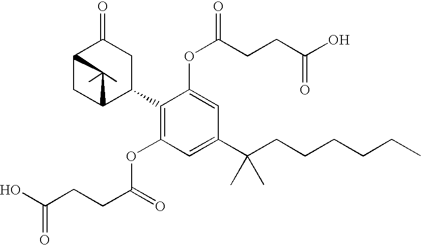 | 572.69 | 18.2 | mg/ml | 5.96 ± 0.41 | 1.07 |
| 18D | 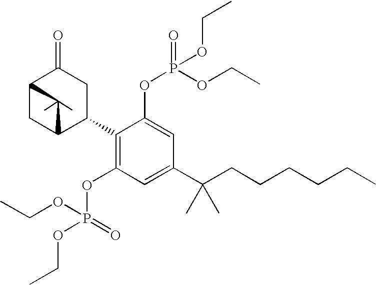 | 644.71 | 2.3 | ng/ml | 7.66 ± 0.50 | 7.66 |
| 18E | 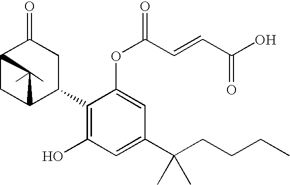 | 444.56 | 0.44 | mg/ml | 5.43 ± 0.40 | 2.69 |
| 18F | 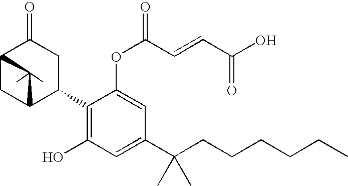 | 470.60 | 0.57 | mg/ml | 6.11 ± 0.48 | 2.46 |
| 18G | 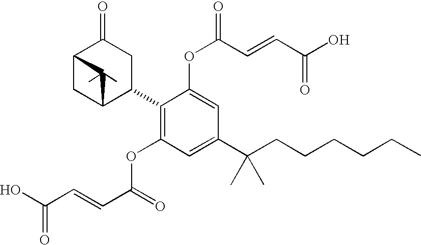 | 568.66 | 54.1 | mg/ml | 5.66 ± 0.60 | 0.66 |
| 18H | 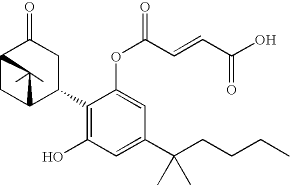 | 458.59 | 10.6 | mg/ml | 5.04 ± 0.40 | 1.39 |
| 18I | 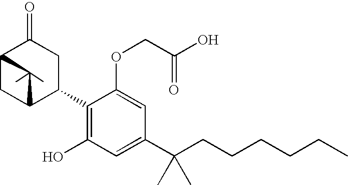 | 430.58 | 0.43 | mg/ml | 6.18 ± .40 | 2.53 |

The actual solubility of certain compounds of the invention was assessed in aqueous buffers and final concentration of compounds was determined using HPLC and spectrophotometer methodologies. It should be noted that compound 18F which according to calculations should have displayed water solubility of about 0.57 mg/ml was found to dissolve in 80 mM phosphate buffer, pH 7.8, at concentration up to 12 mg/ml, about 20-fold higher than expected. For comparison, compound 18B which should have displayed according to the same calculations water solubility of about 24 μg/ml could not be dissolved in aqueous citrate buffer (pH 7.1) at concentration above 95 μg/ml, representing less than a 4-fold divergence from prediction. Therefore, compounds that were designed following the same rational yielded unexpectedly divergent outcome, for instance in connection with solubility in aqueous solutions.

It should be noted that following lyophilization compound 18F could be dissolved in 80 mM phosphate buffer at pH 6.9 even more efficiently at concentrations up to 60 mg/ml, about two order of magnitudes higher than expected.

### Example 11

### Lyophilization

Lyophilization is a drying process in which water or solvent mixtures are removed from a frozen product by sublimation under vacuum. This process is applicable for pharmaceuticals, which are relatively unstable in aqueous solution. Additional advantage of the lyophilization process is that this process can significantly improve the aqueous solubility of synthetic drug substances. Therefore, evaluation of the feasibility of lyophilization process was performed for one preferred compound of the invention.

In order to achieve the best solvent mixture for the dissolution of compound 18F, several experiments were performed and a combination of tert.butanol and water in a ratio of 62.5:32.5 (v/v) was found to be the preferred ratio for dissolution of up to 80 mg/ml of compound 18F in organic cosolvent solution.

A rapid freezing method, i.e. sinking the prepared solution in dry ice, was found to be better than slow freezing in a deep freezer (−30° C.), since a more porous material was obtained by this method. Approximately 11 g of compound 18F were lyophilized in one lyophilization cycle. A cosolvent mixture comprising 38.6% w/v of compound 18F was divided into several dishes to fit the lyophilization chamber. As a result of lyophilization, a dry product with improved physical properties such as flowability and less stickiness was obtained. Solubilization experiments showed that lyophilized compound 18F could be dissolved in 80 mM phosphate buffer up to ˜60 mg/ml if pH was constantly titrated with 0.5N NaOH to value of about 6.9 or above, compared to about 10 mg/ml for non-lyophilized material and to expected calculated value of about 0.6 mg/ml.

### Example 12

### Stability in Aqueous Solutions

Stability of compounds of the invention in aqueous solutions was compared. Compounds were dissolved in buffer phosphate solutions and their concentration monitored using HPLC and spectrophotometer methodology. While compound 18F at concentration of 2 mg/ml in phosphate buffer (pH 7.0) was stable for at least 3 months at 4° C., compound 18B at concentration of 95 μg/ml in citrate buffer (pH 7.1) readily hydrolyzed in less than 24 hours at 4° C. Therefore, compounds that were designed following the same rational yielded unexpectedly divergent outcome, for instance in connection with stability in aqueous solutions.

Evaluation of the therapeutic effects of the orally bioactive cannabinoids of the invention was carried out in a series of experimental systems to support the utility of these drugs as immunomodulatory, anti-inflammatory, analgesic, and neuroprotective agents. These effects were evaluated both in vitro and in vivo, and corroborated utilizing the systems described below. Unless otherwise indicated the test compounds are prepared as follows: for in vitro assays the compounds are first dissolved in DMSO and then stepwise diluted in the assay buffer, generally tissue culture medium, down to a final concentration of 0.1% DMSO. For in vivo assays the test compounds are either (i) first solubilized in CREMOPHOR EL®:ethanol (70% and 30% w/w respectively) and further diluted 1:20 in physiological buffer, generally saline, to reach the appropriate dose; or (ii) solubilized in 80 mM phosphate buffer, pH 7.8 (the final pH of 2 mg/ml stock solutions was brought to pH 6.4 to further increase long term stability). The final molarity for phosphate buffer formulation which achieved optimal solubilization and stability was 68.1 mM HNa₂PO₄.7H₂O and 5.5 mM H₂NaPO₄.2H₂O for buffering properties and 35.5 mM NaCl for isotonicity. HCl was added according to drug concentration to achieve desired pH. Thus, the vehicle control is either the original “solvent” diluted in the appropriate buffer (denoted CE in the case of CREMOPHOR EL®:ethanol) or phosphate buffer pH 6.4 (denoted PB).

All experimentations in animals were performed under humane conditions according to the Israeli Law for Animal Protection—Experiments in Animal 1994. All studies were reviewed by internal ethics committee and approved by the National responsible authority.

### Biological Section

### Example 13

### Binding Affinity for the CB₁ and CB₂ Receptors

The CB₁ and CB₂ binding assays were performed as described in International Patent Application WO 03/063758 and results, expressed as IC₅₀ in nM, are reported in the following table. DMH stands for 1,1-dimethylheptyl, DMP stands for 1,1-dimethylpentyl and DEP stands for diethyl phosphate.

TABLE 2
Substituents and IC₅₀ (nM) of compounds of formula (I)

| | | | | | | | CB₂/ |
|---|---|---|---|---|---|---|---|
| | | | | | | | CB₁ |
| | | | | | | | af- |
| Com- | | | | | CB₂ | CB₁ | finity |
| pound | R₁ | R₂ | R₃ | R₄ | IC₅₀ | IC₅₀ | ratio |
| THC* | | | | | 36.4 | 40.7 | 0.89 |
| 18A | O | OH | OH | DMH | 1 | 27.6 | 28 |
| 18B | O | Succinate | OH | DMH | 1.2 | 41 | 34 |
| 18C | O | Succinate | Succinate | DMH | 1.52 | 117 | 77 |
| 18D | O | DEP | DEP | DMH | >1000 | >1000 | NR |
| 18E | O | Succinate | OH | DMP | 7.4 | 315 | 42 |
| 18F | O | Fumarate | OH | DMH | 9 | 334 | 37.1 |
| 18G | O | Fumarate | Fumarate | DMH | 30 | 325 | 10.8 |

*The binding values of THC are given for comparison and refer to Ki values (nM).

### Example 14

### Effect on MBP Induced Acute EAE

Experimental Autoimmune Encephalomyelitis (EAE), also called Experimental Allergic Encephalomyelitis, is an animal model of Multiple Sclerosis. Various EAE models are known in the art, depending on the method of induction, the strain of the animal and the antigen employed to induce the disease. EAE is an acute or chronic-relapsing, acquired, inflammatory and demyelinating autoimmune disease. Different forms of EAE resemble very closely various forms and stages of MS in a large number of ways.

In the present study, EAE was induced by injection of Myelin Basic Protein (MBP), a method known to model the acute phase of MS. In this model the onset of the disease is observed by the appearance of clinical symptoms about 10 days after induction. The disease progresses and the clinical score increases and peaks around day 15 and spontaneous recovery is observed around day 23 after induction of the disease.

Female Lewis rats (average body weight 130-180 g, Harlan, Israel) were injected s.c. into the hind paws with 25 μg of purified guinea pig myelin basic protein (MBP, Sigma) emulsified in 0.1 ml of Complete Freund's Adjuvant (Difco). Animals were maintained on a 12 hours light/12 hours dark regimen, at a constant temperature of 22° C., with food and water ad libitum. Starting from day 8 following induction, animals were followed up on a daily basis. The results are recorded as clinical score; score of 0 indicates a normal animal with no clinical signs, 0.5 indicates a loss of tonicity in the tail's distal part, 1 indicates whole tail paralysis, 1.5 indicates hind legs weakness in one leg, 2 indicates hind legs weakness in two legs, 2.5 indicates fore legs paralysis in one leg, 3 indicates paralysis of all four legs, 4 indicates complete body paralysis and moribund state and 5 indicates death. The clinical score of the animals is recorded for 15 days following onset of disease until the end of the study 25 days following induction and the area under the curve (AUC) is calculated over this period of time.

Animals that exhibited symptom of the disease, which could be clinically scored between 0.5 and 1, were treated with test compounds or vehicle control for three consecutive days starting from the onset of the disease (˜ at day 9-11 following disease induction). Few routes of administration were assessed and the treatments were either administered intravenously (in CE vehicle) or orally by gavage (in PB vehicle) at volume dose of 5 ml/kg.

On the last day of study (day 25) animals were euthanized with sodium pentobarbitone 100 mg/kg i.p.

Results are expressed as mean±SEM and the differences between the treatment groups were analyzed by analysis of variance (ANOVA) followed by Tukey's post hoc test. A value of p<0.05 was considered to be statistically significant and is indicated on the figure by an asterisk over the relevant treatment group.

Validity of the model was established using methylprednisolone as positive control. When the steroid was administered daily for 5 consecutive days i.v. at 30 mg/kg starting from day of disease induction by MBP injection, a 34% reduction in AUC was reported. The bioactivity of methylprednisolone after disease onset was not established in this study, since it was reported that the steroid is not effective under such conditions in this model.

It was shown that compound 18A yielded a reduction in the AUC of the clinical score in a dose related manner, with a significant reduction of 30-35% at low doses of 0.5-1 mg/kg i.v. Therefore, compounds of the invention administered for 3 days after disease clinical onset are as efficient as methylprednisolone administered for 5 days starting from disease induction before clinical onset. Furthermore, the significant difference in doses needed to achieve the same efficacy should be noted. For prophylactic treatment with methylprednisolone, animals received 5×30 mg/kg, whereas they received only 3×1 mg/kg of compound 18A after clinical onset to achieve similar outcome.

It was also shown that brains and spinal cords removed from treated animals fixed, sectioned and stained using hematoxylin and eosin, displayed significantly less infiltration foci. These observations demonstrated the correlation existing in. this model between the improvement in functional clinical outcome and neuroprotection at histological level in the nervous system.

Compound 18A was dissolved in CREMOPHOR EL®:ethanol and further diluted in physiological buffer prior to i.v. administration. It is now disclosed that compound 18F could be dissolved directly in 80 mM phosphate buffer (pH 6.4) and administered p.o. Compound 18F was administered by oral gavage at doses of 20, 30, 40, 50 and 60 mg/kg to animals showing clinical signs of the disease. Each treatment group comprised at least 10 animals at onset of disease. At some doses, experiments were repeated up to four times.

A low mortality rate was observed in the study (less then 10%) and no differences regarding this parameter were seen among the different treatment groups. Eight percent of vehicle (PB) treated animals died before study completion, whereas this value for the animals receiving 60 mg/kg p.o. of compound 18F is 7%. Moreover, no CB₁ mediated side effects were observed following repeated administration of compound 18F. This observation supports the safety of compounds of the invention.

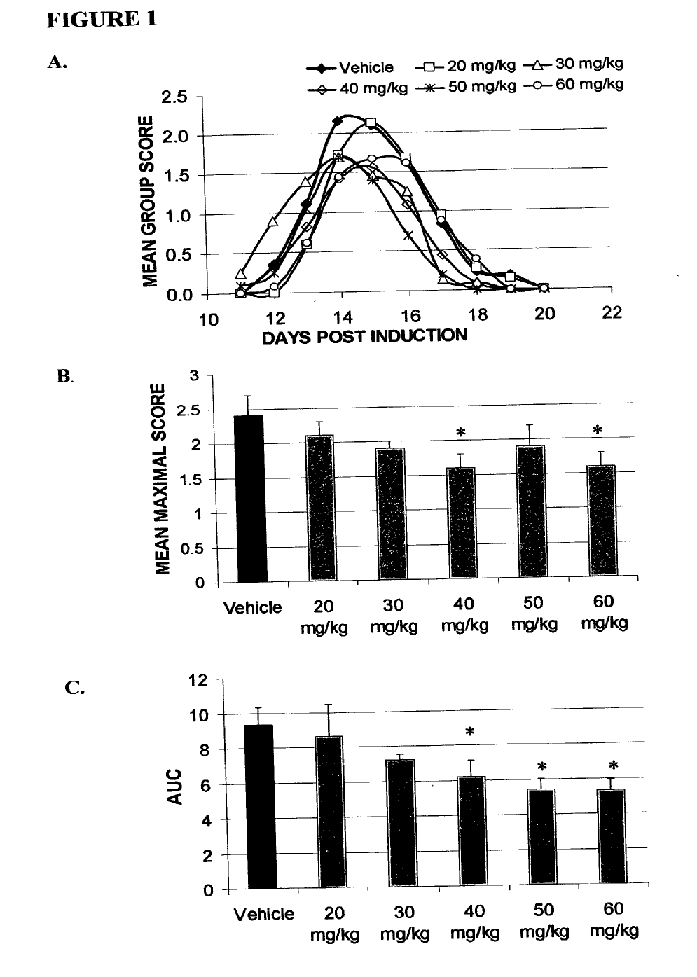

Results are depicted in FIG. 1. Panel A shows the time course of the disease and the effect of p.o. administration of various doses of compound as expressed by the mean group score (MGS) on each study day. While 20 mg/kg p.o. of compound 18F does not significantly affect the clinical outcome, a positive trend of score reduction is already observed at 30 mg/kg, and doses of 40-60 mg/kg seems to display similar efficacy. Decreasing peak of clinical score by close to one point is considered therapeutically significant, especially bearing in mind that maximal clinical score of untreated animals is around 2.2.

The results can be analyzed alternatively by calculating the mean of maximal scores (MMS) obtained by a given treatment group at individual peak of disease generally around days 13-16 following MBP administration. This information is depicted in panel B, where it is clearly appreciable that at 20 mg/kg p.o. only a trend of improvement is observed whereas at doses of 30-60 mg/kg there is a significant reduction of at least 30% in MMS. Finally, the results were analyzed by a third parameter, AUC, which takes into account not only the effect of the treatment at peak of disease, but also along the full disease period till spontaneous remission. Panel C shows the Area Under the Curve of the various treatment groups and by this method the dose related efficacy is best recorded. The best active doses were 50 and 60 mg/kg, which reduced the AUC by 42% and 43% respectively. These effects were statistically significant as compared to the vehicle treated group (p<0.05). The 40 mg/kg reduced the AUC by 33% (p<0.05) and a moderate activity was seen with the 30 mg/kg dose which reduced the AUC by more than 20% (ns).

Altogether, these results demonstrate that compound 18F administered as a treatment (3-4 times, starting on the day of clinical disease manifestation) in an acute EAE model in Lewis rats, reduced the severity of the disease. This activity was expressed by its ability to reduce the disease peak (FIG. 1 panel A), the mean maximal score (FIG. 1 panel B) and the AUC (FIG. 1 panel C). Moreover preliminary results indicate a trend of inhibiting EAE associated pain as measured by mechanical hyperalgesia. In this study a dose of 60 mg/kg p.o. of compound 18F increased the pain threshold over vehicle treated animals by 25%.

There have been reports in the literature of the efficacy of IFN-β in such animal models. For instance, it was shown that administration of IFN-β (Rebif® at dose of 300,000 IU once a day for 3 consecutive days starting from day of MBP injection) reduced the severity of the disease by about 30%. Van Der Meide et al. [Van Der Meide et al., J. Neuroimmunol. 84(1): 14-23, 1998] showed that treatment with IFN-β as a prophylactic starting 2 days before MBP injection and continuing for a total of 10 days, before disease onset, reduced the disease severity by 1 score point at the peak of the disease. These figures are similar to the efficacy achieved by compounds of the invention, but two important points should be emphasized. In these reports IFN-β was administered by injection and before the overt onset of the disease. It has been reported that treatment with IFN-β initiated at a later stage is not effective [Ruuls et al., Immunol. Cell. Biol. 76(1): 65-73, 1998].

For comparison with orally administered cannabinoids, it has been reported that Δ⁸-THC reduced the incidence and severity of neurological deficit in rats inoculated for EAE with autologous spinal cord [Wirguin I. Et al., Immunopharmacology 28(3): 209-14, 1994]. The authors reported that the mean disease severity was reduced from 5.5±0.8 in vehicle treated animals to 4.4±0.8 in Δ⁸-THC treated animals, a statistically significant decrease of 20%. However, it is important to note that Δ⁸-THC, which is the more stable and less psychotropic analog of Δ⁹-THC, displayed this beneficial effect when administered at a dose of 40 mg/kg p.o., starting at time of disease induction, before clinical onset, and continuing daily for 21 consecutive days. Therefore, the cumulative dose needed for efficacy of the reference cannabinoid THC is much higher than for the present compounds.

Thus, compounds of the invention have proven advantage in the treatment of acute peaks of disease, both over IFN-β, which represents the present therapy, since present cannabinoids are effective even administered p.o., and over Δ⁸-THC, which represents the commercially available cannabinoid drug considered for this indication, since they are effective with much less repeated administration. Moreover, compounds of the invention are effective even when administered only 3-4 times after appearance of the disease clinical signs, whereas the two previously mentioned controls, IFN-β and Δ⁸-THC, are beneficial in such models only if frequently administered starting close to disease induction and continuing for repeat administration of ten to twenty-one days.

### Example 15

### Effect on PLP Induced Remitting-Relapsing EAE

As above explained, various inducing agents in different animal species cause the development of slightly different EAE models. While MBP used in Example 14 generates a model wherein a single relapse acute phase of the disease is reproduced, proteolipid protein (PLP) induces a remitting-relapsing type of disorder, which resembles more the initial pattern of neurodeficit outcome in MS patients.

SJCLF1 female mice (6 weeks old, Harlan, Israel) were administered s.c. in three areas (both flanks and the nape of the neck) with 0.2 ml/mouse of emulsified Freund's adjuvant containing 50 μg of PLP and 200 μg of Mycobacterium Tuberculosis. Immediately after, the mice were administered i.v. with 0.1 ml/mouse of phosphate buffer saline (PBS) containing 130 ng of pertussis toxin. This inducing procedure was repeated 48 hours later. Animals were maintained on a 12 hours light/12 hours dark regimen, at a constant temperature of 22° C., with food and water ad libitum. Animals were weighted once a week and clinically evaluated and scored according to the following scoring system: score of 0 indicates a normal animal with no clinical signs, 0.5 indicates a loss of tonicity in the tail's distal part, 1 indicates whole tail paralysis, 1.5 indicates hind legs weakness in one leg, 2 indicates hind legs weakness in two legs, 2.5 indicates fore legs paralysis in one leg, 3 indicates paralysis of all four legs, 4 indicates complete body paralysis and moribund state and 5 indicates death.

The first peak was defined as an increase of at least one score unit sustained for at least two consecutive days after the animal has been injected with the disease inducing agents. Remission was achieved when animals demonstrated a reduction of at least 50% of the peak maximal score and had stabilized to the new score for at least 2 days. Treatment was initiated at peak of first relapse (on day 25) and vehicle (PB) or compound were administered daily for 25 days p.o. by gavage at volume dosage of 5 ml/kg. A third group was composed of untreated animals. Each treatment group comprised 13 mice. Animals were followed for up to two months and during this period two to three minor relapses were observed following the initial first peak of disease.

At the end of the study, mice were euthanized with sodium pentobarbitone, 100 mg/kg i.p. Spinal cords and brains were removed and fixed in 4% formaldehyde solution prior to histological evaluation.

Results are expressed as mean I SEM and the differences between the treatment groups were analyzed by analysis of variance (ANOVA) followed by Tukey's post hoc test. A value of p<0.05 was considered to be statistically significant.

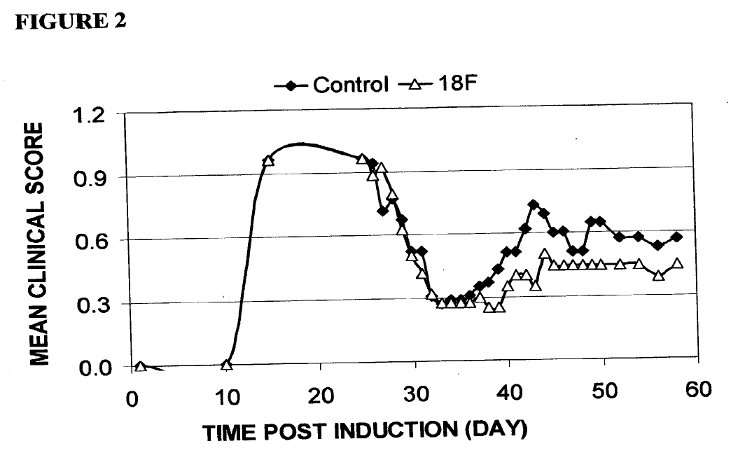

Compound 18F was administered p.o. for 25 days at a dose of 40 mg/kg. No side effects were observed during this period supporting safety of compounds of the invention. Results of clinical score along time are depicted in FIG. 2. Since animals were divided into treatment groups at peak of first relapse, the parameters compared in this study are time to following relapses and amplitude of following peaks. Animals treated with vehicle displayed a pattern essentially similar to untreated animals. The second relapse started at day 35 and 33, and the mean clinical score of the second relapse was 0.7 and 0.8 respectively. Therefore, the average results of these two groups are depicted as control in FIG. 2. Animals treated with compound 18F behaved differently from controls. The clinical score of the second peak was only 0.5 (as compared to about 1 for the first peak) and the relapse was postponed by 5-7 days as compared to controls. When the results are analyzed as percent reduction of clinical score achieved by treatment with compound 18F as compared to control groups, it can clearly be seen that from 13 days after initiation of treatment (day 38) till the end of the study compound 18F reduced the clinical score on average by 28%. This phenomenon was best observed at peaks of relapse, for example at second relapse peak, on days 42-43, compound 18F caused a significant reduction of 35% to 52% in clinical score. Finally, when AUC is calculated for the period spanning from day 35 during first remission to the end of the study (day 58), it is seen that compound 18F caused a highly significant reduction in AUC of 74% as compared to control (AUC_{control}=8.1 vs. AUC_{compound 18F}=2.1; p=0.0025).

Altogether, these results demonstrate that compound 18F administered as a treatment starting from peak of disease in remitting-relapsing PLP induced EAE, reduced the severity of the disease. This activity was expressed by its ability (a) to postpone the recurrence of following relapses, (b) to decrease the mean clinical score of following relapse peak and (c) to reduce the AUC. This study shows that compounds of the invention are effective against various phases of MS. It also further strengthens the efficacy of compounds of the invention by increasing the temporal window wherein administration has beneficial effect. Compounds were previously administered at onset of clinical signs and now at peak of disease. It should be kept in mind that some of the existing MS treatments displayed efficacy is similar models only when administered prophylactically or concurrently with disease induction.

### Example 16

### Effect on MOG Induced Chronic-Progressive EAE

After showing the efficacy of compounds of the invention in EAE models mimicking the acute phase and the remitting relapsing pattern of MS, a third model was established wherein Myelin Oligodendrocyte Glycoprotein (MOG) is used to induce the chronic progressive form of the disease.

C57/BL female mice (6 weeks old, Harlan, Israel) were administered s.c. in two areas in the flank with 0.2 ml/mouse of emulsified Freund's adjuvant containing 200 μg of MOG and 200 μg of Mycobacterium Tuberculosis. This inducing procedure was repeated a week later. Animals were maintained on a 12 hours light/12 hours dark regimen, at a constant temperature of 22° C., with food and water ad libitum. Animals were weighted once a week and clinically evaluated and scored according to the following scoring system: score of 0 indicates a normal animal with no clinical signs, 0.5 indicates a loss of tonicity in the tail's distal part, 1 indicates whole tail paralysis, 1.5 indicates hind legs weakness in one leg, 2 indicates hind legs weakness in two legs, 2.5 indicates fore legs paralysis in one leg, 3 indicates paralysis of all four legs, 4 indicates complete body paralysis and moribund state and 5 indicates death.

Treatment was initiated on day 13 when the animals reached an average clinical score of 0.8 after onset of disease. Vehicle (PB) or compounds (20, 40 and 60 mg/kg of compound 18F) were administered daily for 19 days p.o. by gavage at volume dosage of 5 ml/kg. An additional group was composed of untreated animals. Each treatment group comprised 13 mice. Animals were followed for up to two months following MOG first injection. At the end of the study, mice were euthanized with sodium pentobarbitone, 100 mg/kg i.p. Spinal cords and brains were removed and fixed in 4% formaldehyde solution prior to histological evaluation.

Results are expressed as mean±SEM and the differences between the treatment groups were analyzed by analysis of variance (ANOVA) followed by Tukey's post hoc test. A value of p<0.05 was considered to be statistically significant.

Some minor and transient side effects were observed at high doses during the first 10 minutes post-compound administration. These signs, including minor reduced motor activity, vanished rapidly with chronic treatment and starting from day 4 were totally eliminated.

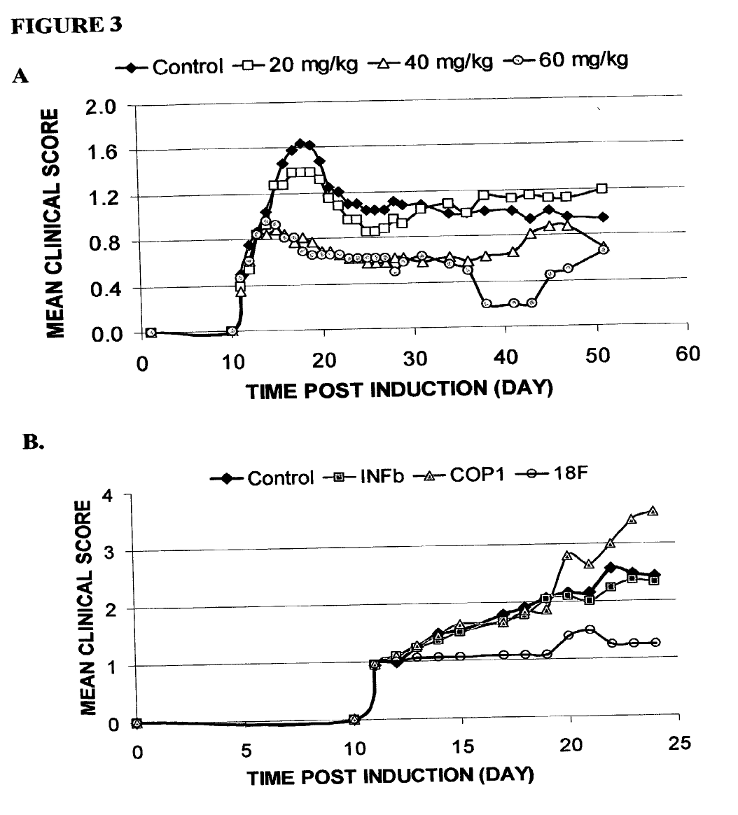

Since animals treated with vehicle displayed a pattern essentially similar to untreated animals, the results of both groups were compiled and are presented hereafter as average control. Results are presented in FIG. 3 panel A, where clinical score for each treatment group is plotted along time. Twenty mg/kg of compound 18F administered p.o. after disease onset have minimal effect and reduce clinical score in the period of the first peak spanning from day 14 to 25 by about 12%. Over the same period, 40 mg/kg and 60 mg/kg significantly and similarly reduce the clinical outcome by 43% and 44%, respectively. The treatment stopped disease progression and no significant peak is observed. The different efficacy of the two doses of compound 18F become apparent later in the progression of the disease, about 20 days after initiation of treatment and after treatment was ceased on day 31. On day 36, sixty mg/kg compound 18F after having stopped disease progression and maintained a plateau level of about 0.6 clinical score (as compared to about 1.2 for control), caused a further marked decrease in disease clinical outcome. For about a week, the animals treated with 60 mg/kg displayed a clinical score of only about 0.2, a reduction of 80% as compared to control at the same period. Animals treated with 40 mg/kg of the compound maintained control over disease progression and plateau level clinical score, but did not cause such a dramatic amelioration of clinical symptoms. It seems that after treatment cessation, animals that received 40 mg/kg gradually loose protection, whereas animals that previously received 60 mg/kg maintain a certain level of protection for a significantly longer period of time. At the end of the study, 20 days after treatment cessation, animals that received 60 mg/kg have on average a clinical score of 0.65 which is still 30% below control at this time (0.95).

Another parameter analyzed was the number of animals that fully recovered at any time point during disease progression, following p.o. administration of compounds of the invention. None of the control animals, either untreated or vehicle treated, recovered from the disease induced in this model. Out of the 13 animals per group, one animal in the 20 mg/kg dose (8%), three in the 40 mg/kg dose (23%) and four in the 60 mg/kg dose (31%) reached a clinical score of 0. Moreover, the time point at which recovery of at least one animal occurred was also dose dependent. At the highest dose, first full recovery was observed on day 16 (i.e. only 3 days after initiation of treatment). At intermediate dose, first full recovery was observed on day 17, while at lowest dose it occurred on day 22.

In a separate study, the efficacy of 40 mg/kg p.o. of compound 18F was compared to existing treatments. COP-1 (Teva, Israel) was administered at 25 mg/kg s.c. and IFN-β (Betaferon, Schering, Germany) at 10,000 IU per mouse s.c. Untreated and vehicle treated animals served as negative controls. Disease was induced as previously described and animals were divided into the various treatment groups when the animals reached an average clinical score of 1. At that time (day 13) treatment was initiated and administered thereafter daily. Animals were followed up for up to one month. At the end of the study, mice were euthanized with sodium pentobarbitone, 100 mg/kg i.p. Spinal cords and brains were removed and fixed in 4% formaldehyde solution prior to histological evaluation.

Since animals treated with vehicle displayed a pattern essentially similar to untreated animals, the results of both groups were compiled and are presented hereafter as average control. Results are presented in FIG. 3 panel B, where clinical score for each treatment group is plotted along time. The disease induced in the present study was severe and its chronic progression is clear with an average clinical score of 1 on day 13 when treatment was initiated and a mean group score of above 2.4 in control untreated or vehicle treated animals on last day. The existing control drugs were not efficacious in this model when administered subcutaneously as treatment of established disease. Animals treated with 10,000 IU IFN-β per day displayed a disease course very similar to control group, whereas animals treated with 25 mg/kg COP-1 developed an apparently more severe outcome with a mean group score of above 3.5 at the end of the study. In this study, 40 mg/kg of compound 18F administered orally significantly stopped disease progression with a mean group clinical score of only 1.25 at the end of the study. This effect is very impressive and statistically significant from day 15 on, i.e. already two days after first treatment. When the results are analyzed as AUC, control group displays a AUC of 24.14 units, IFN-β of 23.08 units and COP-1 of 27.01 units, all strikingly similar. Compound 18F significantly reduced this parameter with AUC of 15.47 units, which is equivalent to a reduction of 36% as compared to control group.

Altogether, the results of Examples 14 to 16 demonstrate that compounds of the invention are efficacious when administered per os in three models of EAE representing various phases of MS in humans. As opposed to existing injectable drugs used in MS treatment, compounds of the invention are effective when administered in established disease after occurrence of clinical signs, either at onset or at peak of disease. These results demonstrate the utility of compounds of the invention in the treatment of disorders having an autoimmune and a neurodegenerative etiology, in particular multiple sclerosis.

### Example 17

### Effect on Biozzi Mice EAE Model

Biozzi mice provide the rodent EAE model closest to human MS. In this study, Biozzi mice are induced to develop EAE by injection of mouse spinal cord tissue. In this model not only can inflammation infiltrates be detected but also demyelination. Moreover, this model allows for the assessment of tremor and spasticity.

Biozzi ABH mice are injected with 1 mg/kg of mouse spinal cord tissue emulsified in Freund's complete adjuvant on days 0 and 7. Animals are followed up for appearance of clinical signs daily following the second inducing injection. Disease induced clinical signs are developed on days 15-20 and are scored as previously described. Spasticity and tremor are assessed visually by blinded analysis according to the method of Baker [Baker D. et al., Nature 404: 84-7, 2000].

Results are expressed as mean±SEM and the differences between the treatment groups are analyzed by analysis of variance (ANOVA) followed by Tukey's post hoc test. A value of p<0.05 is considered to be statistically significant.

### Example 18

### Effect on Gene Expression in CNS of MOG Induced EAE

The purpose of this study was to assess the possible mechanisms underlying the immunomodulatory activity displayed by the CB₂ binding compounds of the invention. It is known that compounds of the invention modulates the secretion of inflammatory agents in activated cells of the immune system, either in vitro or in vivo. In this study the impact on regulation of gene expression was assessed by two methods (a) by real-time RT-PCR and (h) by gene array analysis.

### A—Real-Time RT-PCR

Total RNA is prepared using SV total RNA isolation system (Promega). The brains and spinal cords, which were removed on day 31 from five animals treated with vehicle (i.e. untreated) or 40 mg/kg p.o. of compound 18F (i.e. treated) in the MOG-induced EAE study, were homogenized in lysis buffer. The lysates were transferred to an RNA isolation column, treated with DNAse, washed and eluted according to kit instructions. RNA concentrations were determined using GeneQuant II (Pharmacia-Amersham). Complementary DNA (cDNA) was synthesized from total RNA using SUPERSCRIPT II reverse transcriptase (Life Technologies). 2 μg of total RNA were combined with an oligo (dT)₁₅ primer, 0.5 mM dNTP mix, 8 units of reverse transcriptase and other reaction components up to a final volume of 20 μl, according to the kit instructions. The reaction mixture was incubated at 42° C. for 45 min and inactivated at 70° C. for 15 minutes. Quantitative real-time RT-PCR included 1 μl of the cDNA, 300 nM of the appropriate forward and reverse primers (according to the gene monitored) and 7.5 μl of the reaction mix containing buffer, nucleotides, Taq polymerase and SYBER green (SYBER Green master mix, Applied Biosystems), in a total reaction volume of 15 μl. Gene amplification was obtained using the GeneAmp 5700 sequence detection system (Applied Biosystems). Amplification included one stage of 10 minutes at 95° C. followed by 40 cycles of a 2-steps loop: 20 seconds at 95° C., and 1 minute at 60° C. During each annealing step, the amount of the amplified product was measured by the fluorescence of the double strand DNA binding dye, SYBER Green. The cycle of threshold (C_{T}), representing the PCR cycle at which an increase in fluorescence above a baseline signal can be first detected, was determined for each product. A delay of one PCR cycle in the C_{T} is translated into a two-fold decrease in starting template molecules and vice versa. The changes in the C_{T} of the specific gene product were normalized to the changes in the C_{T} of cyclophilin as reference gene. Results were expressed as fold increase of gene expression in treated or untreated animals above the naive animals. In the following list, the letters [symbol] and [symbol] indicate the forward and reverse primers, respectively.

### Primer Sequences Used:

| | | |
|---|---|---|
| Mouse CB1 [symbol] | 5′-AGACGGTGTTTGCCTTCTGTAGT-3′ | (SEQ ID NO: 1) |
| Mouse GB1 [symbol] | 5′-GCGGAAAGCATGTCTCAGGT-3′ | (SEQ ID NO: 2) |
| Mouse CB2 [symbol] | 5′-GCCTGGGATAGCTCGGATG-3′ | (SEQ ID NO: 3) |
| Mouse GB2 [symbol] | 5′-TGAGAGCCAGTGCAGGGAAC-3′ | (SEQ ID NO: 4) |
| Mouse F4/80 [symbol] | 5′-TTCATCTTGGGCTGCTCCTG-3′ | (SEQ ID NO: 5) |
| Mouse F4/80 [symbol] | 5′-ATTCATCCCGTACCTGACGG-3′ | (SEQ ID NO: 6) |
| Mouse IFN-y [symbol] | 5′-TGAAAATCCTGCAGAGCCAGAT-3′ | (SEQ ID NO: 7) |
| Mouse IFN-y [symbol] | 5′-TGATTCAATGACGCTTATGTTGTTG-3′ | (SEQ ID NO: 8) |
| Mouse IL-1B [symbol] | 5′-ACACTCCTTAGTCCTCGGCCA-3′ | (SEQ ID NO: 9) |
| Mouse IL-1B [symbol] | 5′-CCATCAGAGGCAAGGAGGAA-3′ | (SEQ ID NO: 10) |
| Mouse iNOS [symbol] | 5′-TTCCAGGTGCACACAGGCTA-3′ | (SEQ ID NO: 11) |
| Mouse iNOS [symbol] | 5′-GCACGCTGAGTACCTCATTGG-3′ | (SEQ ID NO: 12) |
| Mouse MCP-1 [symbol] | 5′-TCACAGTTGCCGGCTGG-3′ | (SEQ ID NO: 13) |
| Mouse MCP-1 [symbol] | 5′-TCTTTGGGACACCTGCTGCT-3′ | (SEQ ID NO: 14) |
| Mouse TNF-a [symbol] | 5′-AAGGACTCAAATGGGCTTTCC-3′ | (SEQ ID NO: 15) |
| Mouse TNF-A [symbol] | 5′-CCTCATTCTGAGACAGAGGCAAC-3′ | (SEQ ID NO: 16) |
| Mouse cyclophilin A [symbol] | 5′-TCGCCATTGCCAAGGAGTAG-3′ | (SEQ ID NO: 17) |
| Mouse cyclophilin A [symbol] | 5′-GGTCACCCCATCAGATGGAA-3′ | (SEQ ID NO: 18) |

The results expressed as folds gene expression over naive animals (following normalization to Cyclophilin) are compiled in the following table. By definition the value for naive animals at all instances is one. Statistical significance was analyzed by unpaired two-tailed t-test and one asterisk (*) indicates statistical significance of p<0.05, whereas ** indicates statistical significance of p<0.01.

TABLE 3
Fold gene expression in MOG-induced EAE

| | |
|---|---|
| Brain | Spinal Cord |

| Gene | Untreated | Treated | Untreated | Treated |
|---|---|---|---|---|
| CB₁ | 2 | 1 | 2 | 1 |
| CB₂ | −13 | 1* | 1 | 2 |
| IFN-γ | −9 | 1* | 1.3 | 3 |
| IL-1β | 5.5 | 3* | 11 | 4* |
| TNF-α | 1.5 | 2.5 | 8.5 | 15 |
| MCP-1 | 3 | 5.5 | 19 | 68** |
| F4/80 | 4 | 1.5 | 5.5 | 5.5 |

From the above-table it appears that induction of progressive EAE following MOG injections affect gene expression as recorded 31 days post first injection. Expression of CB₁ is not altered in this model, but all other genes tested showed modifications in fold expression ranging from a 13-fold decrease to a 19-fold increase in untreated animals (vehicle). Though it is generally believed that CB₂ expression might increase in disease state, in the present study it was observed that CB₂ expression decreased by 13 fold in brains of untreated animals. More surprisingly, compound 18F administered p.o. significantly prevented this phenomenon and kept CB₂ expression at normal levels as observed in naive sane animals. Similarly, the level of IFN-γ gene expression was reduced 9-fold in brain of untreated animals and compound 18F significantly prevented this effect, maintaining normal levels of expression. In the brain and spinal cord of untreated animals there was a 5 to 11-fold increase in gene expression of IL-1β. Animals treated p.o. with compound 18F significantly reduced this outcome, bringing back level of expression close to normal with only 3-4-fold overexpression. The level of MCP-1 gene expression increased in both brain and spinal cords of untreated animals. Compound 18F most significantly further increased overexpression in spinal cords from 19-fold to 68-fold. Monocyte chemoattractant protein-1 (MCP-1) is a chemokine of the C—C family, responsible for the recruitment and activation of mainly monocytes, macrophages, basophils, mast cells, T cells, and natural killer (NK) cells. The activated monocytes, which are recruited to the site of injury, secrete in turn inflammatory agents such as TNF-α, IL-1β, nitric oxide and prostaglandins, which at certain levels have beneficiary effects. Neurotrophins are important factors necessary for nerve regeneration. They are abundantly expressed in peripheral nerve tissue and almost absent from the CNS, in correlation with the nerve cell survival and regeneration potential of these respective tissues following injury. Furthermore, it was shown that neurotrophins protect embryonic motoneurons from the deleterious effects of TNF-α and IFN-β [Hammarberg H. et al., Journal of Neuroscience 20(14): 5283-91, 2000]. Immune cells are able to produce neuronal growth factors and the expression of neurotrophins in T and NK cells may therefore be an important mechanism for the protection of CNS neurons from potentially noxious effects of high levels of proinflammatory cytokines.

As recently emphasized by Kotter et al. [Kotter M. R. et al., Neurobiology of Disease 18: 166-75, 2005], though macrophages are mediators of CNS demyelination, they are also implicated in remyelination. Post-mortem evidence from MS tissue as well as experimental findings suggest that remyelination is often associated with areas of robust inflammation and a large macrophage presence. Macrophages might benefit remyelination in two ways. On the one hand they are the main cell type responsible for phagocytic clearance of myelin debris which impair differentiation of oligodendrocyte precursors to pro-remyelinating cells. On the other hand, macrophages are able to secrete a wide variety of factors involved either directly or through signaling in remyelination. In fact the initial inflammatory response triggers a cascade of events which ultimately lead to the creation of a pro-remyelination signaling environment. In this context, the increase in MCP-1 expression observed in this study following per os administration of compound 18F suggests a better recruitment of monocytes in the treated animals and may constitute a protective mechanism by which immune reactions in the CNS do not lead to detrimental effects on nerve cells. In particular, this modulation of inflammatory molecules and their impact on immune cells might create a beneficial pro-remyelinating environment.

In conclusion, these results show for the first time that compounds of the invention protect from progressive EAE by modulating expression of genes involved in the immune system as mediators of inflammation. This modulation is observed 31 days after disease induction, following daily administration of the treatment since disease onset on day 13. This effect of the orally effective cannabinoids of the invention on inflammatory mediators suggests that these compounds might even promote remyelination. Such a process can be beneficial not only for chronic neurodegeneration, whether or not caused by MS, but also for acute forms of demyelination, following for instance spinal cord injury.

### B—Gene Array Analysis

In an additional study, the gene expression profiling was performed using a 400 Gene Array series kit of SuperArray Bioscience Corporation according to manufacturer protocol and RNA extracted from spinal cords of treated or untreated animals, as above described. The scanned data were converted to mRNA expression levels and analyzed using two softwares, ScanAlayze and GEArray Analyzer. The gene array comprised few control genes such as PUC18, GAPDH, CyclophilinA and Beta Actin and all were found to be similarly expressed in vehicle or compound 18F treated animals. Out of the 400 genes tested, twenty were found to be either down-regulated or up-regulated by more than 1.5 fold upon treatment. The genes so identified will be further analyzed by real time RT-PCR at other time points during disease progression. It is interesting to note that the genes whose expression is altered in MOG induced EAE animals following treatment with compounds of the invention broadly speaking encode for proteins involved in the immune system, such as STAT proteins which are involved in signal transduction of several cytokines and growth factors, JAK kinases, β2-microglobulin, TNF-receptor superfamily, calmodulins, and cyclin-dependent kinases.

This second study confirms that compounds of the invention slow the progression of EAE by alteration of gene expression. This activity support that compounds of the invention may be effective in a broad spectrum of disorders wherein gene regulation of similar mediators of inflammation is beneficial.

### Example 19

### Effect on Gene and Protein Expression in Activated Macrophages

This study was designed to assess that the previously reported gene regulation activity correlates with modifications in protein expression and/or secretion. Moreover, this study provides an additional experimental system wherein the anti-inflammatory activity of compounds of the invention is demonstrated.

RAW 264.7 macrophages, a mouse cell line (ATCC # TIB 71), were grown in Dulbecco's modified Eagle's medium (DMEM) with 4 mM L-glutamine adjusted to contain 1.5 g/L sodium bicarbonate, 4.5 g/L glucose, and 10% heat inactivated fetal bovine serum. Cells were grown in tissue culture flasks and seeded at appropriate density into 6 wells tissue culture plates. Four million Raw cells in half a milliliter were stimulated with 1 μg/ml Lipopolysaccharide E. coli 055:B5 (DIFCO Laboratories). The mouse macrophages were pre-treated for one hour with controls or 10 μM of test compounds, and later on activated with LPS. RNA samples were extracted from the cells 3 hrs after activation and iNOS gene expression levels were analyzed by real-time RT-PCR as previously described. For western blot analysis, compounds were tested at 1, 5 and 10 μM and cells were harvested 24 hrs after LPS stimulation. Supernatant were collected and NO secretion was analyzed using ELISA. Cells were washed 3 times with cold PBS, scrapped and lyzed in 100 μl cell lysis buffer containing 20 mM HEPES, pH 7.6, 150 mM NaCl, 1.5 mM MgCl₂, 0.2 mM EDTA, 1% Triton X-100, 10% glycerol, 1 mM DTT, 1 mM PMSF, 10 μg/ml aprotinin. 5 pg/ml leupeptin, 10 mM p-nitrophenyl phosphate, 100 mM β-glycerophosphate, 1 mM sodium fluoride, and 0.1 μM sodium orthovanadate. Cells were lyzed on ice for 10 minutes before further processing.

The results of this experiment are expressed as fold activation of iNOS over non-activated macrophages, after normalization to cyclophilin A expression. Compound 18A inhibits 63% of iNOS gene expression, and compounds 18B, 18E and 18F inhibit 55%, 25% and 100% respectively. It should be noted that the inhibiting activity of compound 18A was reversed by co-administration of either CB₁ or CB₂ antagonists (5 μM of SR-141716 and SR-144528, respectively) but more prominently so by CB₁ antagonists, whereas the inhibiting activity of compound 18F was only blocked by CB₂ antagonists. This surprising observation strengthen the fact that compounds of the invention, though chemically related, have distinct and unexpected properties.

The iNOS gene expression inhibiting activity of compounds of the invention correlated well with the reduction of NO level in supernatant of activated cells. Compounds 18A and 18F inhibited NO secretion in a dose related manner and at 10 μM they yielded 63% and 61% inhibition.

To assess correlation between gene expression and protein expression, protein extracts were prepared from the various treated activated macrophages. Cell lysates were spinned down 10 minutes at 14,000 g at 4° C. Supernatant was collected and prepared for loading onto acrylamide gels according to standard protocols. Electrophoreses proceeded for about 2 hours. Gel was blotted onto nylon membrane, which after blocking was hybridized with primary specific antibodies followed by HRP-labeled secondary antibodies. Primary antibodies against cyclophilin A were used as control (Upstate, Cat# 07-313), and the amount of iNOS was assessed using NOS2 (C11) Santa Cruz antibodies (Cat# SC-7271) were hybridized for 2 hours at room temperature. The secondary antibodies, anti-mouse HRP for iNOS and anti-rabbit HRP for cyclophilin A, were incubated for 1 more hour at room temperature. Following hybridization, the nylon membrane was rinsed, developed with ECL and exposed to film for autoradiography.

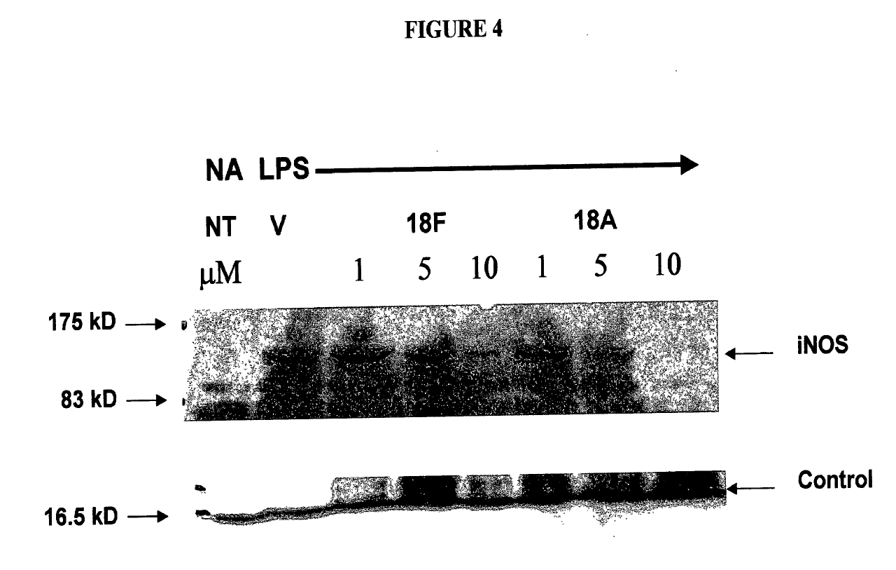

The results of the western blot are depicted in FIG. 4 where a clear dose dependent disappearance of iNOS protein band is observed for both compound 18F and compound 18A. Altogether these results demonstrate that compounds of the invention probably provide part of their immunomodulatory and neuroprotective activities through gene regulation which correlates with alteration of protein levels.

### Example 20

### Analgesic Effect on Visceral Pain

In addition to relapsing-remitting neurological insults, patients suffering from MS also develop additional symptoms such as spasticity and pain. In the present study, the analgesic activity of compounds of the invention was assessed in a model of visceral pain. Visceral pain is caused by disorders of internal organs such as the stomach, kidney, gallbladder, urinary bladder, intestines and others. Visceral pain is nociceptive in nature and believed to be mediated by peritoneal resident cells, such as mast cells and macrophages. Visceral pain usually responds to opioids and NSAIDS. In the present study, the visceral pain was induced in mice by injecting i.p. acetic acid.

Male ICR mice (average body weight 25 g, Harlan, Israel) were pretreated by i.v. injection at volume dose of 5 ml/kg of vehicle, control and test compounds at various doses. For the i.v. part of the study, compounds were dissolved in CREMOPHOR®:Ethanol and diluted 1:20 in saline prior to injection, fifteen minutes before pain induction. When compounds were delivered per os, they were dissolved in phosphate buffer or in CREMOPHOR®:Ethanol as previously described and administered 1 hour before pain induction. Each treatment group, except for controls comprising at least 30 animals, was composed of at least 6 animals. Fifteen or sixty minutes later, depending on the route selected for drug administration, the mice were injected i.p. with 10 ml/kg of 0.6% acetic acid and the number of visceral pain related behaviors (writhing, stretching, contractions of the abdomen accompanied by an elongation of the body and extension of the hind limbs) is counted over a period of 5 minutes, starting 5 minutes after the acetic acid administration. These visceral pain related behaviors are globally defined as writhing responses (WR). The results are expressed as mean number of writhing responses±SEM. Data were analyzed using analysis of variance (ANOVA) followed by post-hoc Fisher test. A value of p<0.05 was considered to be statistically significant and is indicated on the figure by an asterisk over the relevant treatment group.

The following results were obtained at 2 mg/kg i.v. of test compounds or, in the case of the study performed with cannabinoid receptor antagonists, 1 mg/kg test compound or antagonist alone and 1 mg/kg test compound+1 mg/kg antagonist. In a separate study, it was shown that analgesic activity of compounds of the invention was dose related.

Untreated animals displayed on average 28.3±2.5 writhing responses and vehicle only has no effect, with an observed number of writhing responses of 27.6±1.2. The following results are expressed as percent inhibition of writhing responses as compared to vehicle treated group. Two mg/kg of compound 18A was highly analgesic and yielded total inhibition of writhing movements, whereas compounds 18B, 18F and 18G yielded very impressive and highly significant inhibition of 95%, 94% and 91% respectively. Compound 18H reduced by 38% the number of writhing responses, whereas compounds 18D and 18E showed minimal effect at dose tested and fumaric acid at doses up to 5 mg/kg was inactive in this model. Gabapentin at doses up to 200 mg/kg and celecoxib at doses up to 10 mg/kg were inactive in this model.

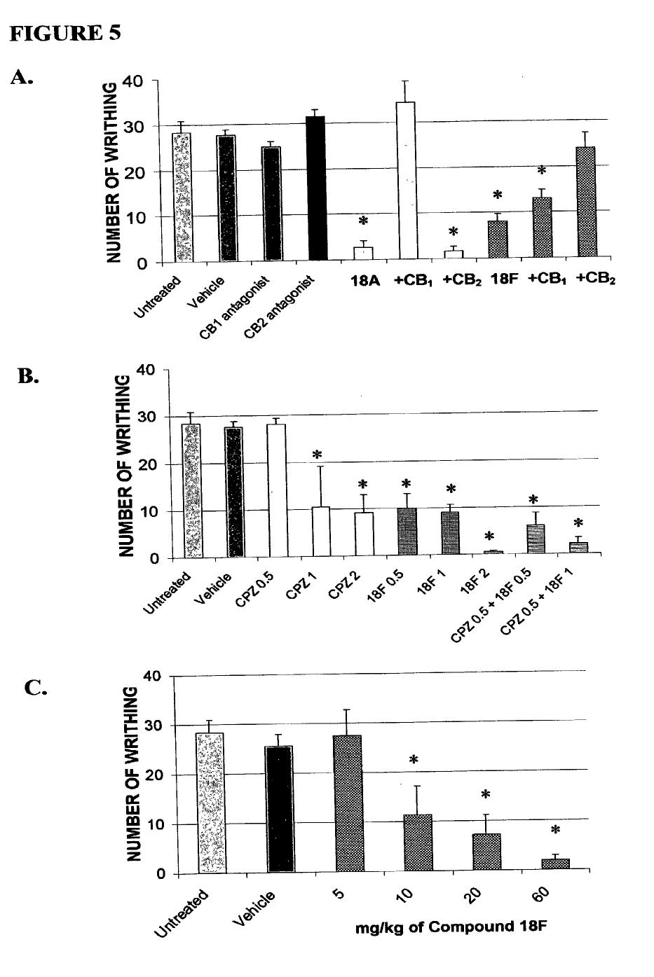

Compounds of the invention were administered i.v. at dose of 1 mg/kg with or without 1 mg/kg CB₁ or CB₂ antagonists, SR141716A and SR144528 respectively. The antagonists were administered separately at same dose, as controls. Results are depicted in FIG. 5 panel A where the mean number of writhing responses (±SEM) of each treatment group is plotted. The controls, Vehicle (CE) and CB₁ or CB₂ antagonists alone, had not effect on the number of writhing responses observed as compared to untreated animals. Compounds 18A and 18F were highly efficient at doses of 1 mg/kg i.v. and inhibited the number of writhing responses by 90% and 71%, respectively. Addition of CB₁ antagonist totally blocked the activity of compound 18A, whereas the activity of compound 18F was only partially reversed and in a non-significant manner. However, addition of CB₂ antagonist did not affect the activity of compound 18A, whereas the activity of compound 18F was significantly reversed. This observation further supports that chemically related compounds of the invention have unexpected divergent mechanisms of action.

In a second study, the visceral pain model was used to assess if compounds of the invention could synergistically act with additional compounds. Capsazepine (CPZ) is a Vanilloid receptor type 1 (VR1) antagonist having analgesic activity. Capsazepine and compound 18F were administered i.v. at 0.5, 1 and 2 mg/kg. Two combination therapy samples were tested, both comprising 0.5 mg/kg of CPZ and either 0.5 or 1 mg/kg of compound 18F. Results are depicted in FIG. 5 Panel B. Both CPZ and compound 18F displayed a dose related reduction of the number of writhing responses. At 0.5 mg/kg, CPZ had no activity, whereas compound 18F already significantly reduced the outcome by 64% as compared to vehicle treated animals. At 2 mg/kg, CPZ significantly reduced the number of writhing responses by 67%, while compound 18F reduced this parameter by 98% almost totally erasing pain response. When inactive dose of 0.5 mg/kg CPZ was combined with either 0.5 or 1 mg/kg of compound 18F, there was a clear trend of enhanced analgesic activity. Combination of the two drugs at 0.5 mg/kg, reduced the number of writhing responses from 10 to 6 (40%), whereas in combination with 1 mg/kg of compound 18F the enhanced activity of the mixture allowed to reduce the number of writhing responses from 9 to 2.2 (75%). These observations support that co-administration or combination of compounds of the invention together with second agents have added advantages. Compounds of the invention could be used alternatively to further enhance the activity of second agents at safe doses or to reduce the dose of second agents to levels where said agents would be safe and associated, if at all, with less side effects, while maintaining original therapeutic activity of the previous higher dose of second agent.

In a third study, compounds of the invention were administered p.o. in phosphate buffer at increasing doses ranging from 5 to 60 mg/kg. Results are depicted in FIG. 5 Panel C. Compound 18F reduced the number of writhing responses in a dose dependent manner which was significant starting from 10 mg/kg and reached 93% inhibition at the highest dose tested.

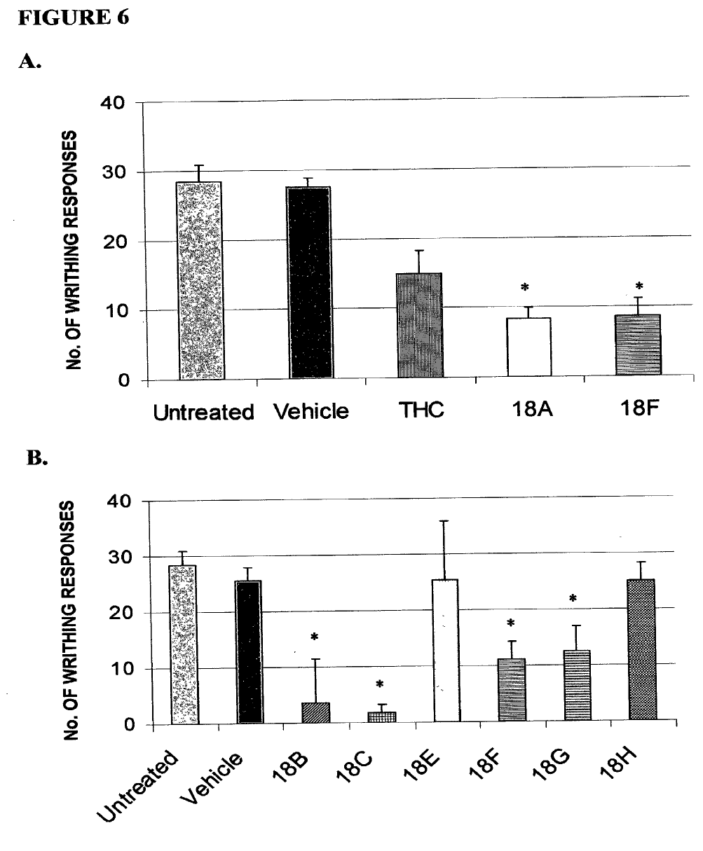

Finally, after having established that efficacy per os was already significant at 10 mg/kg, various compounds of the invention were tested at this single dose following oral route of administration. In the first part of the study, the compounds were dissolved in CREMOPHOR®:Ethanol to allow comparison with Δ⁸-THC which is not soluble in aqueous buffer. Results are depicted in FIG. 6 panel A. Though, 10 mg/kg Δ⁸-THC decrease the number of writhing responses by 47% as compared to the untreated group, due to variability this reduction is not statistically significant as compared to vehicle treated animals. On the other hand, the orally effective cannabinoids of the invention have at the same dose significantly decreased the pain response, as expressed by 70% and 71% inhibition of writhing responses in animals treated with compounds 18A and 18F respectively.

After having established that the orally effective cannabinoids of the invention are superior to THC control following oral administration in cosolvent vehicle, the water-soluble compounds were dissolved in phosphate buffer. Results are depicted in FIG. 6 panel B. Out of the six compounds tested, four were significantly analgesic following oral administration of 10 mg/kg. Compounds 18B, 18C, 18F, and 18G inhibited the pain response as compared to untreated animals by 88%, 93%, 61%, and 56%, respectively. Compounds 18E and 18H were not effective at the dose tested. In is interesting to note that compound 18H was previously found active when administered intravenously. These observations support that cannabinoids of the invention are effective compounds, in the present case as analgesics, following oral administration.

### Example 21

### Analgesic Effect on Inflammatory Pain

The purpose of this study is to test the anti-inflammatory pain activity of the compounds. Inflammatory pain is nociceptive in nature, wherein the pain sensation is often perceived for longer period than in acute pain such as elicited in Example 20. Wherein in visceral pain, the prophylactic analgesic activity of the compounds was assessed for up to about one hour, in the present model the duration of the preventive activity of compounds against acute pain was assessed for up to about three hours. Inflammatory pain and paw edema were induced by injection of 2% λ carrageenan in the animal hind paw.

Male Sprague Dawley rats (average body weight 200 g, Harlan, Israel) were transiently sedated by placement on dry ice for the duration of the injections. Rats were injected subcutaneously, in the subplantar region of one (right) paw with 0.1 ml of 2% w/v λ Carrageenan in sterile saline. The contralateral (left) paw was not injected as data from the literature, confirmed by our own experience, showed that injection of 0.1 ml of normal saline did not affect later analgesic measurements. Test compounds were administered p.o. by oral gavage immediately after the carrageenan injection as pretreatment. Vehicle (PB) treated animals were used as controls. Before induction of inflammatory pain and three hours after injection, the animals reactions to pain stimuli were tested in two systems. The first stimulus was thermal and assessed by the Plantar Test according to Hargreaves, using Ugo Basile Model 7370. The scale was set to an intensity of 50 arbitrary units. The latency time till the animal lift a paw as a reaction to the thermal stimulus was recorded for both the inflamed and non-inflamed hind paws. The second stimulus was mechanical (tactile) and assessed using a Dynamic Plantar Sesthesiomether (Ugo Basile Model 73400-002). The system was set on maximal force of 50 grams and the force applied was gradually increased at the rate of 10 g/sec. Finally, the impact on paw edema was assessed. Paw thickness is measured using a dial thickness gauge (Spring-dial, constant low pressure gauge, Mitutoyo, TG/L-1, 0.00 mm) and paw volume is measured using a plethysmometer (model #7150, Ugo Basile, Italy). At the end of the study, animals were euthanized with an i.p. injection of 100 mg/kg pentobarbitone.

The results are measured as the differences between the two hind paws at time 0 and 3 hours both as ALT, for the latency time in the thermal part of the study, and as ΔForce, for the mechanical part of the study. The paw volume is expressed as percent from vehicle treated animals. Results are expressed as mean±SEM for each treatment group and the differences among those groups are analyzed by analysis of variance (ANOVA) followed by post-hoc Tukey's test. A value of p<0.05 was considered to be statistically significant and is indicated on the figure by an asterisk over the relevant treatment group.

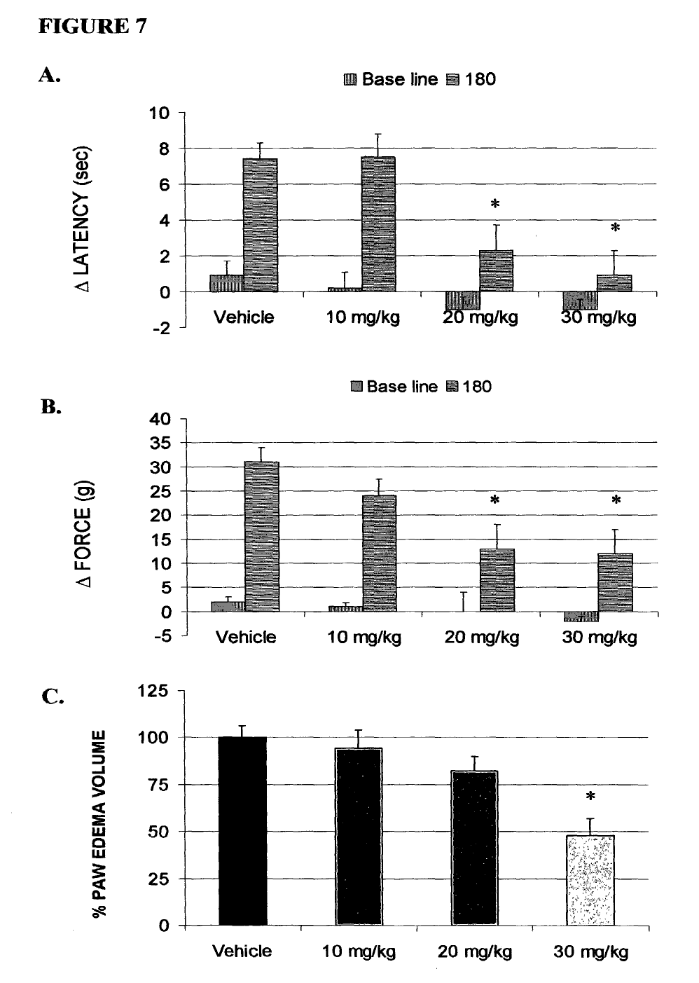

Administration of 2% λ carrageenan induced localized and transient paw inflammation, characterized by swelling and redness of the paws. It was shown that compound 18F when administered p.o. by oral gavage at doses of 10, 20 and 30 mg/kg, immediately after the carrageenan injection, displayed preventive analgesic activity. Three hours after pain induction, animals treated with vehicle only displayed a ΔLT of about 7.5 seconds between the hind paws following thermal stimulus. Results are depicted in FIG. 7 panel A. This outcome was reduced in a dose related manner following oral administration of compound 18F. Animals receiving 10 mg/kg displayed a ΔLT of 7.5 seconds (i.e. no reduction), whereas at 20 and 30 mg/kg this outcome dropped down to latency of 2.3 and 0.9 sec (i.e. a reduction of 69% and 88%, respectively). Animals treated with vehicle only displayed, when the stimulus applied was tactile, a delta force between paws of 31 g. Results are depicted in FIG. 7 panel B. The force required to cause the rat to lift their paws was reduced in a dose dependent manner by 7 when 10 mg/kg of compound 18F was administered p.o. (i.e. 23% reduction), and by 18 and 19 g for the higher doses (i.e. about 60% reduction in pain threshold for 20 and 30 mg/kg p.o.). Finally, the impact on the localized inflammatory component of the model was assessed by measuring paw volume of the various treatment groups. By definition, vehicle treated animals have 100% of maximal paw volume. Results are depicted in FIG. 7 Panel C. This parameter was also dose related and 10 mg/kg of compound 18F caused a slight decrease of 6% in paw volume, whereas 20 mg/kg yielded a reduction of 18% and 30 mg/kg a significant reduction of 52%.

These results demonstrate that compound 18F is a potent prophylactic analgesic and local anti-inflammatory agent, when administered per os concomitantly with acute pain induction.

It is now disclosed and emphasized that compound 18G which differs from compound 18F by a further substitution of the remaining phenolic hydroxyl with fumarate is not active in this model when administered p.o. at single dose of 20 mg/kg. At 20 mg/kg the paw volume of animals treated with compound 18F was 82% of vehicle, whereas animals treated with compound 18G were unaffected by treatment with 96% of control paw volume. When thermal stimulus was applied the ALT was reduced by 69% by compound 18F, whereas compound 18G was ineffective in lowering this parameter. Finally, when mechanical stimulus was applied, animals treated with compound 18F displayed 58% reduction in Δforce, while compound 18G only slightly and non-significantly affected this parameter and lowered it by 8%. Unexpectedly, bi-substitution of the phenolic hydroxyls does not yield a further improved compound as compared to its active mono-substituted counterpart. On the contrary, in this model of acute pain the bi-substitution seems to be deleterious further supporting the fact that the present findings concerning preferred compounds of the invention are not obvious.

### Example 22

### Analgesic effect on Neuropathic Pain

Among the various types of pain symptoms MS patient can develop, neuropathic pain is predominant. Neuropathic pain, associated with chronic pain, differs from previously assessed visceral and inflammatory pain, associated with acute pain. Acute pain and chronic pain differ in their etiology, pathophysiology, diagnosis and treatment. Acute pain is nociceptive in nature and occurs secondary to chemical, mechanical and thermal stimulation of A-delta and C-polymodal pain receptors. Acute pain is self-limiting and will vanish on short-term after initial injury. Chronic pain, on the other hand, is continuous and can persist for years after the initial injury. It is produced by damage to, or pathological changes in the peripheral or central nervous system. Neuropathic pain tends to be only partially responsive to opioid therapy. Drugs active against certain types of acute pain such as visceral pain and inflammatory pain are therefore not necessarily effective against neuropathic pain.

The analgesic activity of compounds of the invention was assessed in two models of neuropathic pain: (a) chronic constriction induced (CCI) and (b) Taxol® induced.

### A—Chronic Constriction Induced Neuropathic Pain

A peripheral monopathy was induced in the right hind limb of rats following a chronic constriction of the sciatic nerve according to Bennet et al. [Bennet, G. J. & Xie, Y-K., Pain 33: 87-107, 1988]. The development of mechanical allodyna was monitored using an established behavioral test (Von Frey filaments).

Pre-surgery baseline values were ascertained as the mean of 2 pre-surgery values. Once the baseline values had been established, the animals were surgically prepared by constricting the right sciatic nerve with 4 chromic cat gut loose ligatures. On day 11 post-operation, the animals that have developed mechanical allodyna were arbitrarily allocated to the various treatment groups based on the pre-surgery values.

The design was randomized, performed in a masked fashion as to whether drug or vehicle is being given. The animals, male Sprague-Dawley rats (average body weight 240-290 g, Harlan, Israel), were allowed to acclimatize to the behavioral testing equipment prior to testing. On the testing day, the animals, at least six per treatment group, were given p.o. various doses of compound 18F by gavage with a volume of 5 ml/kg. The study included vehicle (PB) treated negative control and morphine-treated (5 mg/kg s.c) positive control. Fifteen minutes later, a series of Von Frey filaments (pre-calibrated prior to testing) were applied to the plantar surface of the hind paw, from below. The filaments were applied in ascending order starting with the weakest force and the withdrawal threshold for both the ipsilateral and contralateral hind paws was evaluated. Each filament was indented on the mid-plantar surface of the foot to the point where it just starts to bend; this is repeated approximately 8-10 times per filament at a frequency of approximately 1 Hz. The withdrawal threshold is defined as being the lowest force of two or more consecutive Von Frey's filaments to elicit a reflex withdrawal response (i.e. a brief paw flick) and is measured in grams.

Results are expressed as mean±SEM for each treatment group and the differences among those groups are analyzed by analysis of variance (ANOVA) followed by post-hoc Tukey's test. A value of p<0.05 was considered to be statistically significant and is indicated on the figure by an asterisk over the relevant treatment group.

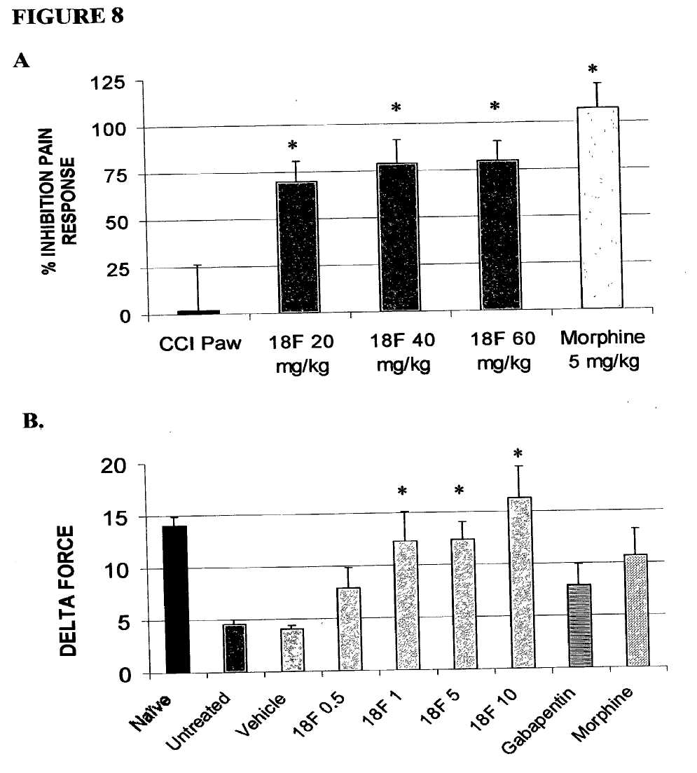

Results are depicted in FIG. 8 panel A. Compound 18F had little effect at 20 mg/kg p.o. on neuropathic pain. But oral administration of higher doses has significantly reduced the pain response in the operated limbs, as compared to pre-dosing responses. Forty mg/kg of compound 18F given orally was as strong as 5 mg/kg morphine administered subcutaneously. Results were thereafter analyzed as percent inhibition of Pain Response as calculated by (ΔLT_{Vehicle}−ΔLT_{Treatment})/ΔLT_{Vehicle}.

### B—Taxol® Induced Neuropathic Pain

Taxol® (Paclitaxel) is one of the most effective and commonly used anti-neoplastic drugs for the treatment of solid tumors. It has two serious side effects: myelosuppression and peripheral neuropathy. Granulocyte colony-stimulating factor effectively counteracts the neutropenia in most patients. But there are no acceptable therapies to prevent or minimize the nerve damage, making neurotoxicity a significant dose-limiting side effect. Clinically, paclitaxel-induced neurotoxicity is presented as a sensory neuropathy, with the most common complaints being numbness, tingling and burning pain. These signs start usually in the legs and seen later in the hands. The incidence of Taxol® induced neuropathy in clinic is on average 30%. In the present study, neuropathic pain was induced using Taxol® according to the method of Polomano et al. [Polomano R. C. et al., Pain 94: 293-304, 2001].

Male Sprague Dawley rats (average body weight 150-200 g, Harlan, Israel) were administered i.p. with 6 mg/kg Taxol® (Bristol Myers Squibb, USA) every other day for 9 days. On day 13, baseline pain threshold was measured. One hour later, the animals were administered with 5 ml/kg i.p. of vehicle (PB) only and pain threshold was re-established. After an additional hour animals were treated with various doses of compound 18F ranging from 0.5 mg/kg up to 10 mg/kg administered i.p. Morphine at 5 mg/kg i.p. and gabapentin at 100 mg/kg i.p served as positive controls. Each treatment group comprised at least eight animals. One hour post drug administration pain threshold was monitored using thermal and mechanical stimuli as previously described. At the end of the study, rats were euthanized with sodium pentobarbitone 100 mg/kg i.p. Sample rats were then transfused with saline and heparinized 4% formaldehyde solution. The L4-L5 area of the spinal cord and the sciatic nerve were taken for histopathology assessment.

The time latencies for thermal hyperalgesia, and force needed to induce pain of the different treatment groups are compared, using one way ANOVA (analysis of variance), followed by Duncan's post hoc test. A p<0.05 value was considered statistically significant and is indicated on the figure by an asterisk over the relevant treatment group.

Results are displayed in FIG. 8 panel B as Δforce per treatment group. Taxol® induced neuropathy reduced the pain threshold on day 13 from 15 gm baseline value of naive animals to 5 gm in untreated animals. Administration of vehicle 5 ml/kg i.p. did not affect the pain threshold. Treatment with compound 18F increased the pain threshold in a dose-related manner. The 1, 5 and 10 mg/kg doses demonstrated values statistically significant from the vehicle treated animals. The 10 mg/kg dose increased the pain threshold back to pre-Taxol® baseline. Both positive controls, gabapentin 100 mg/kg and morphine 5 mg/kg, showed a trend of increasing the pain threshold. The effect induced by both of them was not statistically significant (while compared to the vehicle-treated group). The increase in pain threshold by compound 18F at 10 mg/kg i.p. was statistically different (p<0.05) from the effect exerted by either gabapentin or morphine.

As previously explained, neuropathic pain is produced by damage to, or pathological changes in the peripheral or central nervous systems, and can be found not only in association with MS but in other numerous pathologies, especially involving chronic non-malignant pain. The ability of compounds of the invention to reduce neuropathic pain has therefore wide beneficial impact and support that compounds of the invention may advantageously replace or supplement existing treatments.

In addition compounds of the invention may be administered to patient receiving anti-neoplastic therapy having neuropathic side-effects, such as Taxol®. The present study has clearly established that compound of the invention have neuroprotective effect and prevent the neurological side effects of Taxol® chemotherapy.

### Example 23

### Safety

Cannabinoids despite their impressive therapeutic potential are hardly found in medical use primarily due to legal concern. Though the psychoactive cannabimimetic effects are not addictive and are in certain circumstances out-weighted by their therapeutic benefice, for most legislators cannabis is a drug and its bioactive components or derived products should be banned. The development of cannabinoid drugs is therefore accompanied by added safety concern. As explained the psychoactive cannabimimetic effects are mediated through the CB₁ receptor, and therefore CB₂ selective compounds are a priori safer drug candidates. For comparison, the approved Δ⁹-THC has a CB₂/CB₁ affinity ratio of 0.89 whereas compounds of the invention have at least 10-fold better affinity and more generally about 30-fold more selectivity toward CB₂. Residual CB₁ related activities, if any, were assessed in the Tetrad Assay wherein impact of compounds on the body temperature, spontaneous and forced locomotor activity and catalepsy were measured.

ICR male mice (average body weight 25 g, Harlan, Israel) were administered by oral gavage at volume dose of 5 ml/kg various doses of compound 18F, namely 30, 60, 80 and 100 mg/kg. The following measurements were made pre-dosing to establish baseline and 30 minutes, 3 hours and 24 hours after administration. Rectal temperature was monitored using a thermistor probe (YSI model 400, USA). Spontaneous locomotion was assessed using the open field methodology. The animal walking distance and speed were recorded and analyzed during a period of three minutes using a video camera connected to a computerized system. At the end of each open field test, the animals were tested for catalepsy symptoms. This was carried out by gently forcing the animal to stand on its hind paws when its front paws are holding on an elevated beam. The time for the animal to step down of the beam was measured in seconds. A normal animal withdraws the beam immediately whereas cataleptic animal tend to stay on the beam. The longer the animal stays leaning on the beam the more cataleptic the animal is. For the calculation of percentage of cataleptic animals per group a cut off of 5 seconds was determined (i.e. an animal that leaned on the beam for more than 5 sec was considered cataleptic animal). Animal coordination and motor activity under forced condition was evaluated using the accelerated rotarod performance test. The animals were trained for 4 days before beginning the experiment. Their task was to stay on the accelerating rod without falling for 12 minutes (3 minutes at each speed). The tested speeds were: 15, 19, 23 and 27 rpm. Animal performance on the rod was scored as follows: each animal could obtain a maximum of 3 points (1 for each minute) for full walking on the rod at each speed. Therefore, an animal could get a maximum score of 12 points (3 for each speed). Catching the circling beam of the rod without walking subtracted 0.5 points for every 3 circles circled by the animal. The first 3 circles did not affect the score.

At the end of the study the animals were killed using an injection of 100 mg/kg pentobarbital.

Compound 18F p.o. had no effect on three of the motor related parameters monitored: catalepsy and either spontaneous or forced locomotor activity, up to maximal dose tested of 100 mg/kg. The sole parameter affected was body temperature, where administration of compound 18F resulted in transient hypothermia. Normothermia was recovered in a dose-dependent manner. At lower doses of 30 and 60 mg/kg, animals regained normothermia within 24 hours, whereas animals administered the higher doses of 80 and 100 mg/kg were still 2° C. below baseline at the end of follow-up period. Overall, no behavioral side effects were observed during the course of the study and at therapeutically effective doses of up to 60 mg/kg the compound was found to be safe.

Although the present invention has been described with respect to various specific embodiments presented thereof for the sake of illustration only, such specifically disclosed embodiments should not be considered limiting. Many other such embodiments will occur to those skilled in the art based upon applicants' disclosure herein, and applicants propose to be bound only by the spirit and scope of their invention as defined in the appended claims.

1-52. (canceled)

53. A method of alleviating or treating multiple sclerosis comprising the step of orally administering to an individual in need thereof an effective amount of a pharmaceutical composition comprising as an active ingredient a compound of formula (I): 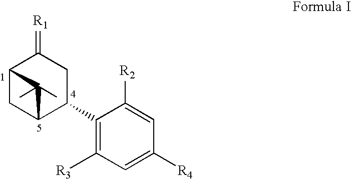
having a specific stereochemistry wherein C-4 is S, the protons at C-1 and C-5 are cis in relation to one another and the protons at C-4 and C-5 are trans; and wherein:
R₁ is selected from the group consisting of
(a) O or S,
(b) C(R′)₂ wherein R′ at each occurrence is independently selected from the group consisting of hydrogen, cyano, —OR″, —N(R″)₂, a saturated or unsaturated, linear or branched C₁-C₆ alkyl, C₁-C₆ alkyl-OR″ and C₁-C₆ alkyl-N(R″)₂ wherein at each occurrence R″ is independently selected from the group consisting of hydrogen, C(O)R′″, C(O)N(R′″)₂, C(S)R′″, saturated or unsaturated, linear or branched C₁-C₆ alkyl, C₁-C₆ alkyl-OR′″, and C₁-C₆ alkyl-N(R′″)₂, wherein at each occurrence R′″ is independently selected from the group consisting of hydrogen or saturated or unsaturated, linear, branched or cyclic C₁-C₁₂ alkyl, and
(c) NR″ or N—OR″ wherein R″ is as previously defined;
R₂ and R₃ are each independently selected from the group consisting of
(a) —R″, —OR″, —N(R″)₂, —SR″, —S(O)(O)NR″, wherein at each occurrence R″ is as previously defined,
(b) —S(O)R^{b}, —S(O)(O)R^{b} wherein R^{b} is selected from the group consisting of hydrogen, saturated or unsaturated, linear or branched C₁-C₆ alkyl, C₁-C₆ alkyl-OR″ and C₁-C₆ alkyl-N(R″)₂, wherein R″ is as previously defined, and
(c) —OC(O)OH, —OS(O)(O)OR^{e}, —OP(O)(OR^{e})₂, —OR^{d} or —OC(O)—R^{d} chain terminated by —C(O)OH, —S(O)(O)OR^{e}, or —P(O)(OR^{e})₂, wherein R^{d} is a saturated or unsaturated, linear or branched C₁-C₆ alkyl and R^{e} is at each occurrence selected from the group consisting of hydrogen and R^{d} as previously defined; and
R₄ is selected from the group consisting of
(a) R wherein R is selected from the group consisting of hydrogen, halogen, OR′″, OC(O)R′″, C(O)OR′″, C(O)R′″, OC(O)OR′″, CN, N(R′″)₂, NC(O)R′″, NC(O)OR′″, C(O)N(R′″)₂, NC(O)N(R′″)₂, and SR′″, wherein at each occurrence R′″ is as previously defined,
(b) a saturated or unsaturated, linear, branched or cyclic C₁-C₁₂ alkyl-R wherein R is as previously defined,
(c) an aromatic ring which can be further substituted at any position by R wherein R is as previously defined, and
(d) a saturated or unsaturated, linear, branched or cyclic C₁-C₁₂ alkyl which, at its terminus, is unsubstituted or substituted by an aromatic ring which is unsubstituted or substituted as defined in (c);
and pharmaceutically acceptable salts, esters or solvates thereof.

54. The method of claim 53, wherein R₁ is O, R₂ and R₃ are each OR^{f} wherein at each occurrence R^{f} is independently selected from the group consisting of hydrogen, —R^{d} and —C(O)—R^{d}, wherein R^{d} is a saturated or unsaturated, linear or branched C₁-C₆ alkyl chain terminated by —C(O)OR^{g} and R^{g} is selected from the group consisting of hydrogen and a saturated or unsaturated, linear or branched C₁-C₆ alkyl, and R₄ is selected from the group consisting of a saturated or unsaturated, linear, branched or cyclic C₁-C₁₂ alkyl-R^{h} wherein R^{h} is selected from the group consisting of R and an aromatic ring which is unsubstituted or substituted at any position by R as previously defined.

55. The method of claim 54, wherein R₁ is O, R₂ and R₃ are each independently selected from the group consisting of OH, succinate, fumarate, and methylenoxycarboxyl, and R₄ is selected from the group consisting of 1,1-dimethylpentyl, 1,1-dimethylheptyl, 1,1-dimethyl-6-heptynyl, 1,1-dimethyl-3-phenyl-propyl, 1,1,3-trimethyl-butyl, 1-(4-chloro-phenyl)-1-methyl-ethyl, 1-ethyl-1-methyl-propyl, 5-bromo-1,1-dimethylpentyl and 1,1-dimethyl-pent-4-enyl.

56. The method of claim 55, wherein R₁ is O, R₂ is OH, R₃ is fumarate and R₄ is 1,1-dimethylheptyl.

57. The method of claim 53, wherein the pharmaceutical composition further comprises a pharmaceutically acceptable carrier, diluent or excipient.

58. The method of claim 53, wherein the active ingredient of formula (I) is administered at daily dose of about 0.05 to about 50 mg per kg body weight, in a regimen of 1, 2, 3 or 4 times a day.

59. The method of claim 53, wherein the pharmaceutical composition is administered orally by peroral, mucosal, buccal, gingival, lingual, sublingual or oropharyngeal administration.

60. The method of claim 59, wherein the pharmaceutical composition is administered in liquid, aerosol or solid unit dosage form selected from solutions, suspensions, micelles, emulsions, microemulsions, aerosols, powders, granules, sachets, soft gels, tablets, pills, caplets and capsules.

61. The method of claim 53, further comprising co-administering the compound of formula (I) with one or more second agents which are independently selected from the group consisting of compounds of formula (I), immunomodulators, immunosuppressors, steroids, anti-convulsants, analgesics, anti-depressants, muscle relaxants, anti-spasticity agents, anti-tremor-agents, tricyclic antidepressants, non steroidal anti-inflammatory drugs (NSAID), selective serotonin reuptake inhibitors (SSRI), monoamine oxidase inhibitors (MOI), antidepressants, benzodiazepines (BZD), anticholinergic agents, beta blockers, laxatives, and channel blockers.

62. The method of claim 61, wherein co-administration of the therapeutic agents is performed in a regimen selected from: a single combined composition, separate individual compositions administered substantially at the same time, and separate individual compositions administered under separate schedules.

63. The method of claim 62, wherein the second agent is independently selected from the group consisting of IFN-β, IFN-β-1a, IFN-β-1b, glatiramer acetate, azathioprine, cladribine, cyclophosphamide, mitoxantrone, prednisone and methylprednisolone.

64. A method of alleviating or treating neurological symptoms selected from the group consisting of tremor, spasticity, muscle weakness, and lack of coordination, comprising the step of orally administering to an individual in need thereof an effective amount of a pharmaceutical composition comprising as an active ingredient a compound of formula (I): 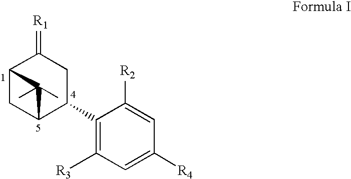
having a specific stereochemistry wherein C-4 is S, the protons at C-1 and C-5 are cis in relation to one another and the protons at C-4 and C-5 are trans; and wherein:
R₁ is selected from the group consisting of
(a) O or S,
(b) C(R′)₂ wherein R′ at each occurrence is independently selected from the group consisting of hydrogen, cyano, —OR″, —N(R″)₂, a saturated or unsaturated, linear or branched C₁-C₆ alkyl, C₁-C₆ alkyl-OR″ and C₁-C₆ alkyl-N(R″)₂ wherein at each occurrence R″ is independently selected from the group consisting of hydrogen, C(O)R′″, C(O)N(R′″)₂, C(S)R′″, saturated or unsaturated, linear or branched C₁-C₆ alkyl, C₁-C₆ alkyl-OR′″, and C₁-C₆ alkyl-N(R′″)₂, wherein at each occurrence R′″ is independently selected from the group consisting of hydrogen or saturated or unsaturated, linear, branched or cyclic C₁-C₁₂ alkyl, and
(c) NR″ or N—OR″ wherein R″ is as previously defined;
R₂ and R₃ are each independently selected from the group consisting of
(a) —R″, —OR″, —N(R″)₂, —SR″, —S(O)(O)NR″, wherein at each occurrence R″ is as previously defined,
(b) —S(O)R^{b}, —S(O)(O)R^{b} wherein R^{b} is selected from the group consisting of hydrogen, saturated or unsaturated, linear or branched C₁-C₆ alkyl, C₁-C₆ alkyl-OR″, and C₁-C₆ alkyl-N(R″)₂, wherein R″ is as previously defined, and
(c) —OC(O)OH, —OS(O)(O)OR^{e}, —OP(O)(OR^{e})₂, —OR^{d} or —OC(O)—R^{d} chain terminated by —C(O)OH, —S(O)(O)OR^{e}, or —P(O)(OR^{e})₂, wherein R^{d} is a saturated or unsaturated, linear or branched C₁-C₆ alkyl and R^{e} is at each occurrence selected from the group consisting of hydrogen and R^{d} as previously defined; and
R₄ is selected from the group consisting of
(a) R wherein R is selected from the group consisting of hydrogen, halogen, OR′″, OC(O)R′″, C(O)OR′″, C(O)R′″, OC(O)OR′″, CN, N(R′″)₂, NC(O)R′″, NC(O)OR′″, C(O)N(R′″)₂, NC(O)N(R′″)₂, and SR′″, wherein at each occurrence R′″ is as previously defined,
(b) a saturated or unsaturated, linear, branched or cyclic C₁-C₁₂ alkyl-R wherein R is as previously defined,
(c) an aromatic ring which can be further substituted at any position by R wherein R is as previously defined, and
(d) a saturated or unsaturated, linear, branched or cyclic C₁-C₁₂ alkyl which, at its terminus, is unsubstituted or substituted by an aromatic ring which is unsubstituted or substituted as defined in (c);
and pharmaceutically acceptable salts, esters or solvates thereof.

65. The method of claim 64, wherein R₁ is O, R₂ and R₃ are each OR^{f} wherein at each occurrence R^{f} is independently selected from the group consisting of hydrogen, —R^{d} and —C(O)—R^{d}, wherein R^{d} is a saturated or unsaturated, linear or branched C₁-C₆ alkyl chain terminated by —C(O)OR^{g} and R^{g} is selected from the group consisting of hydrogen and a saturated or unsaturated, linear or branched C₁-C₆ alkyl, and R₄ is selected from the group consisting of a saturated or unsaturated, linear, branched or cyclic C₁-C₁₂ alkyl-R^{h} wherein R^{h} is selected from the group consisting of R and an aromatic ring which is unsubstituted or substituted at any position by R as previously defined.

66. The method of claim 65, wherein R₁ is O, R₂ and R₃ are each independently selected from the group consisting of OH, succinate, fumarate, and methylenoxycarboxyl, and R₄ is selected from the group consisting of 1,1-dimethylpentyl, 1,1-dimethylheptyl, 1,1-dimethyl-6-heptynyl, 1,1-dimethyl-3-phenyl-propyl, 1,1,3-trimethyl-butyl, 1-(4-chloro-phenyl)-1-methyl-ethyl, 1-ethyl-1-methyl-propyl, 5-bromo-1,1-dimethylpentyl and 1,1-dimethyl-pent-4-enyl.

67. The method of claim 66, wherein R₁ is O, R₂ is OH, R₃ is fumarate and R₄ is 1,1-dimethylheptyl.

68. The method of claim 64, wherein the pharmaceutical composition further comprises a pharmaceutically acceptable carrier, diluent or excipient.

69. The method of claim 64, wherein the active ingredient of formula (I) is administered at daily dose of about 0.05 to about 50 mg per kg body weight, in a regimen of 1, 2, 3 or 4 times a day.

70. The method of claim 64, wherein the pharmaceutical composition is administered orally by peroral, mucosal, buccal, gingival, lingual, sublingual or oropharyngeal administration.

71. The method of claim 70, wherein the pharmaceutical composition is administered in liquid, aerosol or solid unit dosage form selected from solutions, suspensions, micelles, emulsions, microemulsions, aerosols, powders, granules, sachets, soft gels, tablets, pills, caplets and capsules.

72. The method of claim 64, further comprising co-administering the compound of formula (I) with one or more second agents which are independently selected from the group consisting of compounds of formula (I), anti-convulsants, analgesics, anti-depressants, muscle relaxants, anti-spasticity agents, anti-tremor-agents, tricyclic antidepressants, non steroidal anti-inflammatory drugs (NSAID), selective serotonin reuptake inhibitors (SSRI), monoamine oxidase inhibitors (MOI), antidepressants, benzodiazepines (BZD), anticholinergic agents, beta blockers, laxatives, and channel blockers.

73. The method of claim 72, wherein co-administration of the therapeutic agents is performed in a regimen selected from: a single combined composition, separate individual compositions administered substantially at the same time, and separate individual compositions administered under separate schedules.

74. A method of modulating mediators of inflammation comprising the step of orally administering to an individual in need thereof an effective amount of a pharmaceutical composition comprising as an active ingredient a compound of formula (I): 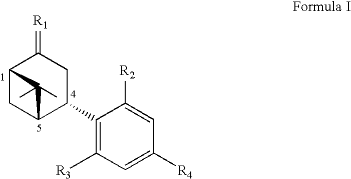
having a specific stereochemistry wherein C-4 is S, the protons at C-1 and C-5 are cis in relation to one another and the protons at C-4 and C-5 are trans; and wherein:
R₁ is selected from the group consisting of
(a) O or S,
(b) C(R′)₂ wherein R′ at each occurrence is independently selected from the group consisting of hydrogen, cyano, —OR″, —N(R″)₂, a saturated or unsaturated, linear or branched C₁-C₆ alkyl, C₁-C₆ alkyl-OR″ and C₁-C₆ alkyl-N(R″)₂ wherein at each occurrence R″ is independently selected from the group consisting of hydrogen, C(O)R′″, C(O)N(R′″)₂, C(S)R′″, saturated or unsaturated, linear or branched C₁-C₆ alkyl, C₁-C₆ alkyl-OR′″, and C₁-C₆ alkyl-N(R′″)₂, wherein at each occurrence R′″ is independently selected from the group consisting of hydrogen or saturated or unsaturated, linear, branched or cyclic C₁-C₁₂ alkyl, and
(c) NR″ or N—OR″ wherein R″ is as previously defined;
R₂ and R₃ are each independently selected from the group consisting of
(a) —R″, —OR″, —N(R″)₂, —SR″, —S(O)(O)NR″, wherein at each occurrence R″ is as previously defined,
(b) —S(O)R^{b}, —S(O)(O)R^{b} wherein R^{b} is selected from the group consisting of hydrogen, saturated or unsaturated, linear or branched C₁-C₆ alkyl, C₁-C₆ alkyl-OR″, and C₁-C₆ alkyl-N(R″)₂, wherein R″ is as previously defined, and
(c) —OC(O)OH, —OS(O)(O)OR^{e}, —OP(O)(OR^{e})₂, —OR^{d} or —OC(O)—R^{d} chain terminated by —C(O)OH, —S(O)(O)OR^{e}, or —P(O)(OR^{e})₂, wherein R^{d} is a saturated or unsaturated, linear or branched C₁-C₆ alkyl and R^{e} is at each occurrence selected from the group consisting of hydrogen and R^{d} as previously defined; and
R₄ is selected from the group consisting of
(a) R wherein R is selected from the group consisting of hydrogen, halogen, OR′″, OC(O)R′″, C(O)OR′″, C(O)R′″, OC(O)OR′″, CN, N(R′″)₂, NC(O)R′″, NC(O)OR′″, C(O)N(R′″)₂, NC(O)N(R′″)₂, and SR′″, wherein at each occurrence R′″ is as previously defined,
(b) a saturated or unsaturated, linear, branched or cyclic C₁-C₁₂ alkyl-R wherein R is as previously defined,
(c) an aromatic ring which can be further substituted at any position by R wherein R is as previously defined, and
(d) a saturated or unsaturated, linear, branched or cyclic C₁-C₁₂ alkyl which, at its terminus, is unsubstituted or substituted by an aromatic ring which is unsubstituted or substituted as defined in (c);
and pharmaceutically acceptable salts, esters or solvates thereof.

75. The method of claim 74, wherein the modulated mediators of inflammation are selected from the group consisting of inflammatory related genes, cytokines, chemokines, cannabinoid receptors, STAT signal transducers, JAK kinases, microglobulins, TNF-α and receptor superfamily, calmodulins, cyclin dependent kinases, CB₂, IL-β, IFN-γ, iNOS and MCP-1.

76. The method of claim 75, wherein R₁ is O, R₂ and R₃ are each OR^{f} wherein at each occurrence R^{f} is independently selected from the group consisting of hydrogen, —R^{d} and —C(O)—R^{d}, wherein R^{d} is a saturated or unsaturated, linear or branched C₁-C₆ alkyl chain terminated by —C(O)OR^{g} and R^{g} is selected from the group consisting of hydrogen and a saturated or unsaturated, linear or branched C₁-C₆ alkyl, and R₄ is selected from the group consisting of a saturated or unsaturated, linear, branched or cyclic C₁-C₁₂ alkyl-R^{h} wherein R^{h} is selected from the group consisting of R and an aromatic ring which is unsubstituted or substituted at any position by R as previously defined.

77. The method of claim 76, wherein R₁ is O, R₂ and R₃ are each independently selected from the group consisting of OH, succinate, fumarate, and methylenoxycarboxyl, and R₄ is selected from the group consisting of 1,1-dimethylpentyl, 1,1-dimethylheptyl, 1,1-dimethyl-6-heptynyl, 1,1-dimethyl-3-phenyl-propyl, 1,1,3-trimethyl-butyl, 1-(4-chloro-phenyl)-1-methyl-ethyl, 1-ethyl-1-methyl-propyl, 5-bromo-1,1-dimethylpentyl and 1,1-dimethyl-pent-4-enyl.

78. The method of claim 77, wherein R₁ is O, R₂ is OH, R₃ is fumarate and R₄ is 1,1-dimethylheptyl.


SEQUENCE LISTING (18 sequences)

SEQ ID NO: 1 - DNA, 23 bp, Artificial sequence
  Single strand DNA oligonucleotide
agacggtgtttgccttctgtagt

SEQ ID NO: 2 - DNA, 20 bp, Artificial sequence
  Single strand DNA oligonucleotide
gcggaaagcatgtctcaggt

SEQ ID NO: 3 - DNA, 19 bp, Artificial sequence
  Single strand DNA oligonucleotide
gcctgggatagctcggatg

SEQ ID NO: 4 - DNA, 20 bp, Artificial sequence
  Single strand DNA oligonucleotide
tgagagccagtgcagggaac

SEQ ID NO: 5 - DNA, 20 bp, Artificial sequence
  Single strand DNA oligonucleotide
ttcatcttgggctgctcctg

SEQ ID NO: 6 - DNA, 20 bp, Artificial sequence
  Single strand DNA oligonucleotide
attcatcccgtacctgacgg

SEQ ID NO: 7 - DNA, 22 bp, Artificial sequence
  Single strand DNA oligonucleotide
tgaaaatcctgcagagccagat

SEQ ID NO: 8 - DNA, 25 bp, Artificial sequence
  Single strand DNA oligonucleotide
tgattcaatgacgcttatgttgttg

SEQ ID NO: 9 - DNA, 21 bp, Artificial sequence
  Single strand DNA oligonucleotide
acactccttagtcctcggcca

SEQ ID NO: 10 - DNA, 20 bp, Artificial sequence
  Single strand DNA oligonucleotide
ccatcagaggcaaggaggaa

SEQ ID NO: 11 - DNA, 20 bp, Artificial sequence
  Single strand DNA oligonucleotide
ttccaggtgcacacaggcta

SEQ ID NO: 12 - DNA, 21 bp, Artificial sequence
  Single strand DNA oligonucleotide
gcacgctgagtacctcattgg

SEQ ID NO: 13 - DNA, 17 bp, Artificial sequence
  Single strand DNA oligonucleotide
tcacagttgccggctgg

SEQ ID NO: 14 - DNA, 20 bp, Artificial sequence
  Single strand DNA oligonucleotide
tctttgggacacctgctgct

SEQ ID NO: 15 - DNA, 21 bp, Artificial sequence
  Single strand DNA oligonucleotide
aaggactcaaatgggctttcc

SEQ ID NO: 16 - DNA, 23 bp, Artificial sequence
  Single strand DNA oligonucleotide
cctcattctgagacagaggcaac

SEQ ID NO: 17 - DNA, 20 bp, Artificial sequence
  Single strand DNA oligonucleotide
tcgccattgccaaggagtag

SEQ ID NO: 18 - DNA, 20 bp, Artificial sequence
  Single strand DNA oligonucleotide
ggtcaccccatcagatggaa